(19) United States
(12) Patent Application Publication
(10) Pub. No.: US 2008/0268529 A1
(43) Pub. Date: Oct. 30, 2008

(54) Container for Nucleic Acid Amplification, Nucleic Acid Preparation Kit and Nucleic Acid Analyzer

(75) Inventors: Noriaki Furusato, Kyoto (JP); Koji Hirayama, Kyoto (JP); Shinya Nakajima, Kyoto (JP); Daisuke Takahashi, Kyoto (JP); Shinichi Ohta, Kyoto (JP); Atsushi Murakami, Kyoto (JP)

Correspondence Address: HAMRE, SCHUMANN, MUELLER & LARSON, P.C., P.O. BOX 2902, MINNEAPOLIS, MN, 55402-0902, US

(73) Assignee: ARKRAY, INC., Kyoto-shi, Kyoto (JP)

(21) Appl. No.: 11/628,399

(22) PCT Filed: Jun. 1, 2005
(86) PCT No.: PCT/JP05/10080

§ 371 Date: Dec. 1, 2006

(30) Foreign Application Priority Data
Jun. 2, 2004 (JP) 2004-164987
Jun. 2, 2004 (JP) 2004-164988
Jun. 2, 2004 (JP) 2004-164989
Feb. 10, 2005 (JP) 2005-034775

(51) Int. Cl. C12M 1/00

(52) U.S. Cl. 435/289.1

(57) ABSTRACT

The present invention relates to a technique of amplifying a target nucleic acid contained in a specimen, and further to a technique of analyzing the amplified target nucleic acid. The present invention provides a nucleic acid amplification container (3) to be installed in a nucleic acid analyzing apparatus when used. The nucleic acid amplification container (3) includes a container main body (30) having a reactor (34) where the target nucleic acid and an amplification reagent are to be reacted, and a cap (31) that covers an upper opening of the reactor (34) and can be completely separated from the container main body (30).

### TECHNICAL FIELD

The present invention relates to a technique of amplifying target nucleic acid contained in a specimen, and further to a technique of analyzing the amplified target nucleic acid.

### BACKGROUND ART

Analysis of nucleic acid, which has been playing an important role in the medical field for genetically diagnosing an infection or genetic disorder, is currently applied to various fields such as agriculture and foodstuffs, in addition to the medical field. Generally, the analysis of nucleic acid is executed through such processes as purification of the nucleic acid from a specimen, amplification of the purified nucleic acid, and detection of the amplified nucleic acid. From the viewpoint of manpower cost, reproducibility and analyzing efficiency, it is preferable that the respective process is automatically executed by machinery, and ideally it is desirable that all the processes are automatically executed by machinery (for example, refer to patent documents 1, 2).

Attempts for automating the nucleic acid purification include employing a nucleic acid binding carrier. An example of such methods employs a nucleic acid binding silica particle and chaotropic ion (for example, refer to patent document 3). The method includes mixing with a specimen the nucleic acid binding silica particle and the chaotropic ion capable of releasing the nucleic acid in the specimen to bond the nucleic acid in the specimen to the nucleic acid binding silica particle, isolating the solid phase and the liquid phase, and eluting the nucleic acid bonded to the nucleic acid binding silica particle. For isolating the solid phase and the liquid phase, however, centrifugation or filtration with a filter has to be performed, which complicates the operation and structure of the machinery when the automation is realized.

Another method of employing the nucleic acid binding carrier employs a magnetic carrier (for example, refer to patent documents 4, 5) The method includes causing a magnetic silica particle to adsorb the nucleic acid, isolating the silica particle with a magnet, eluting the nucleic acid from the isolated silica particle and then collecting the eluate. This method does not require such operation as centrifugation for isolating the solid phase and the liquid phase, and is hence advantageous in automating the process with machinery.

However, this method provides a relatively low collection rate of the nucleic acid, and besides the collection rate is prone to fluctuate depending on the type of the sample. Moreover, it has been discovered that the magnetic silica particle acts as an inhibitor against the amplification reaction, when a polymerase chain reaction (PCR) process is adopted for the nucleic acid amplification. In addition, popular detection methods of the nucleic acid include providing a sign on the nucleic acid and optically measuring the sign, however when such method is adopted the magnetic silica incurs a measurement error, and fluctuation in content of the magnetic silica particle during the purification process degrades the reproducibility.

Meanwhile, the PCR process is a typical example of the method of amplifying the nucleic acid, in which the amplification of the nucleic acid by the PCR process is sufficiently automated and the PCR apparatuses are already commercially available. Those PCR apparatuses are generally designed not only for amplifying the nucleic acid but also for detecting the amplified nucleic acid.

When employing one of the commercially available PCR apparatuses, however, an exclusive amplification kit has to be used. Popular amplification kits contain a primer and a reagent such as polymerase, prepared in advance in a container with a cap. Accordingly, the user is compelled to carry out the operations of opening the cap of the container, dispensing the nucleic acid solution in the container, agitating the reacting solution in the container and closing the cap, and then setting the container in the PCR apparatus. Thus, the popular amplification kit largely depends on the manual operation by the user thereby imposing a burden on the user, and besides the dependence on the manual operation by the user leads to lower analyzing efficiency, as well as degradation in reproducibility due to a difference in skill among the individual users. Also, the container employed in the amplification kit is usually a general-purpose article integrally formed with the cap by a resin molding process, and hence it is difficult for the PCR apparatus to automatically open and close the cap. For these reasons, as long as employing the popular amplification kit, it is difficult to automatically execute with the PCR apparatus the operation so far manually performed by the user.

Further, in a popular analyzing apparatus such as a nucleic acid analyzing apparatus, a pipet apparatus for dispensing liquids such as reagents and specimen is incorporated. The pipet apparatus includes a nozzle which is, depending on the type of the analyzing apparatus, horizontally or vertically movable by a robot arm (for example, refer to patent document 6). On the other hand, for automating the nucleic acid analyzing apparatus, other elements of the pipet apparatus than the nozzle may have to be movably set inside the analyzing apparatus. In this case, the plurality of movable elements including the nozzle have to be incorporated in the nucleic acid analyzing apparatus such that those elements do not interfere one another, and that those movable elements are independently driven under control. Incorporating thus the plurality of movable elements in the nucleic acid analyzing apparatus incurs an increase in dimensions of the nucleic acid analyzing apparatus, and in manufacturing cost thereof.

Patent document 1: JP-A-2001-149097

Patent document 2: JP-A-2003-304899

Patent document 3: JP-B-2680462

Patent document 4: JP-A-S60-1564

Patent document 5: JP-A-H09-19292

Patent document 6: JP-A-2002-62302

### DISCLOSURE OF THE INVENTION

An object of the present invention is to automate the series of processes for analyzing nucleic acid, including purification of the nucleic acid, amplification of the nucleic acid and measurement thereof, thereby alleviating the burden of the user and improving the analyzing efficiency and reproducibility.

Another object of the present invention is to automate the analysis by the nucleic acid analyzing apparatus, without incurring an increase in dimensions of the apparatus, and in manufacturing cost thereof.

A first aspect of the present invention provides a nucleic acid amplification container to be set in a nucleic acid analyzing apparatus. The container comprises a container main body including a reactor in which a target nucleic acid is to be reacted with an amplification reagent, and a cap that covers an upper opening of the reactor and is removably attached to the container main body.

A second aspect of the present invention provides a nucleic acid preparation kit to be set in a nucleic acid analyzing apparatus. The kit comprises a nucleic acid extracting container used for extracting a target nucleic acid from a specimen, and a nucleic acid amplification container that amplifies the target nucleic acid. The nucleic acid amplification container includes a container main body including a reactor in which the target nucleic acid is to be reacted with an amplification reagent, and a cap that covers an upper opening of the reactor and is removably attached to the container main body.

In the first and the second aspect of the present invention, the cap may be thread-engageable with the reactor and removable from and attachable to the reactor by applying a rotational force. In the case where the nucleic acid analyzing apparatus includes a rotating member that applies the rotational force to the cap, the cap may include an engaging portion to be engaged with the rotating member thus to enable the rotating member to apply the rotational force.

The engaging portion may include a column-shaped recessed portion in which the rotating member is inserted, and the recessed portion may include a plurality of vertically extending ribs circumferentially aligned on an inner circumferential surface at regular intervals. It is preferable that the rib has a reducing width toward an upper end portion thereof.

The cap may include a projection via which the rotating member retains the cap. The projection may be formed as an outwardly projecting flange.

The nucleic acid extracting container may include a nucleic acid extracting element that extracts the target nucleic acid from the specimen and carries the extracted nucleic acid, and a container main body formed as a separate body from the nucleic acid extracting element and including an accommodation chamber that stores therein the nucleic acid extracting element.

It is preferable that the nucleic acid extracting element and the cap are provided with a retaining device that causes the cap to retain the nucleic acid extracting element cap to integrally move the nucleic acid extracting element with the cap.

The retaining device may include a protruding or recessed portion for engagement provided on one of the nucleic acid extracting element and the cap, and one or more engaging pawls provided on the other of the nucleic acid extracting element and the cap, to be engaged with the protruding or recessed portion for engagement.

It is preferable that the nucleic acid extracting element and the cap are provided with a guide mechanism that delimits a position of the cap with respect to the nucleic acid extracting element, when the cap is caused to retain the nucleic acid extracting element. The guide mechanism may include a pin provided on one of the nucleic acid extracting element and the cap, and an insertion hole provided on the other of the nucleic acid extracting element and the cap, for the pin to be inserted therein.

The nucleic acid extracting element may include a solid matrix that carries the target nucleic acid, and a retaining member that retains the solid matrix.

It is preferable that the nucleic acid amplification container is disposed such that the solid matrix is spaced from a bottom portion of the reactor when the nucleic acid extracting element is taken out of the accommodation chamber and accommodated in the reactor.

The retaining member may include a sealing member that defines a sealed space in the reactor, when the nucleic acid extracting element is accommodated in the reactor while being retained by the cap. In this case, the sealing member is fixed at an upper position than where the solid matrix is retained.

The retaining member may include a projection to be engaged with a stepped portion of the reactor, and the projection may be formed as an outwardly projecting flange.

When the nucleic acid analyzing apparatus includes a transferring member that takes out the nucleic acid extracting element from the accommodation chamber and transfers the nucleic acid extracting element to the reactor, the retaining member may include an engaging portion to be engaged with the transferring member, and the projection may be formed to disengage the transferring member and the retaining member. When the nucleic acid analyzing apparatus further includes a cylindrical member that encloses the transferring member and relatively movable in a vertical direction with respect to the transferring member, the projection is formed such that a downward force is exerted thereto by interference by the cylindrical member that takes place when the cylindrical member is relatively moved downward with respect to the transferring member.

The solid matrix may be retained by the retaining member with an inclination with respect to a vertical axis of the retaining member, and more preferably, in a horizontal or generally horizontal orientation with respect to the vertical axis. In this case, it is preferable to form the solid matrix in a disk shape.

The inclination of the solid matrix with respect to the vertical axis may be achieved, for example, by piercing the solid matrix with the retaining member. In this case, the retaining member may include a tapered portion with a reducing diameter toward an end portion, a pin-shaped portion extending from the tapered portion to penetrate through the solid matrix, and a stopper piece that restricts the solid matrix from coming off from the pin-shaped portion.

Also, the solid matrix may be retained by the retaining member in a parallel or generally parallel orientation with respect to the retaining member. In this case, it is preferable to form the solid matrix in a sheet-shape.

The parallel or generally parallel orientation of the solid matrix with respect to the vertical axis may be achieved by suspending the solid matrix from the retaining member. In this case, the retaining member may include a holder that holds an end portion of the solid matrix to suspend the solid matrix.

In the nucleic acid preparation kit according to the present invention, the nucleic acid extracting container may further include one or more cleaner wells that store therein a cleaning liquid for removing impurity other than the target nucleic acid from the nucleic acid extracting element, and the nucleic acid amplification container may further include one or more reagent wells that store therein such reagents that may be required for amplifying the target nucleic acid.

A third aspect of the present invention provides a nucleic acid amplification apparatus for use with a nucleic acid amplification container installed therein, wherein the nucleic acid amplification container includes a container main body including a reactor in which the target nucleic acid is to be reacted with an amplification reagent, and a cap that covers an upper opening of the reactor, and that can be completely separated from the container main body.

A fourth aspect of the present invention provides a nucleic acid analyzing apparatus for use with a nucleic acid extracting container and a nucleic acid amplification container to prepare a target nucleic acid from a specimen and to analyze the target nucleic acid, wherein the nucleic acid amplification container includes a container main body including a reactor that provides a space for amplifying the target nucleic acid with a nucleic acid extracting element retaining the target nucleic acid extracted from the specimen, and a cap that covers an upper opening of the reactor.

It is preferable that the nucleic acid analyzing apparatus according to the present invention further includes a cap attaching/removing device that attaches and removes the cap. In the nucleic acid analyzing apparatus, the nucleic acid amplification container may be configured to employ the cap that is screw-engaged with the reactor, so that exerting a rotational force to the cap allows attaching/removing the cap to and from the reactor. In this case, the cap attaching/removing device also includes a rotating member that applies the rotational force to the cap.

In the nucleic acid analyzing apparatus, the nucleic acid amplification container may be configured to employ the cap that includes an engaging portion having a column-shaped recessed portion in which a tip portion of the rotating member is inserted, and a plurality of vertically extending ribs circumferentially aligned at regular intervals on an inner circumferential surface of the recessed portion. In this case, the rotating member may include a plurality of protrusions to be located between adjacent ones of the plurality of ribs of the cap when the tip portion is inserted in the recessed portion, and the plurality of protrusions may be formed to vertically extend with a reducing width toward a lower end portion.

In the nucleic acid analyzing apparatus, the nucleic acid amplification container may be configured to employ the cap that includes a projection formed to project outward. In this case, the cap attaching/removing device may include an engaging pawl to be engaged with the projection, and moves the cap at least in a vertical direction, with the engaging pawl being engaged with the projection.

When the cap of the nucleic acid amplification container is set to retain the nucleic acid extracting element of the nucleic acid extracting container, the cap attaching/removing device operates to move the cap taken out of the reactor, cause the cap to retain the nucleic acid extracting element so far retained in the accommodation chamber, thereby taking out the nucleic acid extracting element from the accommodation chamber and moving the cap with the nucleic acid extracting element to accommodate the nucleic acid extracting element in the reactor, and then to cover the upper opening of the reactor with the cap.

When using the nucleic acid amplification container configured to employ the cap that includes the recessed portion and the flange, the cap attaching/removing device may include a fitting element to be fitted in the recessed portion, and a cylindrical element that encloses the fitting element and includes a pawl portion to be engaged with the flange.

The nucleic acid analyzing apparatus according to the present invention may include a transferring member that takes out the nucleic acid extracting element from the accommodation chamber and transfers the nucleic acid extracting element to the reactor.

In a preferred embodiment, the nucleic acid analyzing apparatus further includes a cylindrical member that encloses the transferring member and is relatively movable in a vertical direction with respect to the transferring member. The cylindrical member removes the nucleic acid extracting element coupled with the transferring member, when moved downward with respect thereto.

The nucleic acid analyzing apparatus according to the present invention may further include a control unit that controls a movement of the transferring member and the cap attaching/removing device. The control unit executes the steps of causing the rotating member retaining the cap to retreat from right above the reactor after removing the cap from the reactor with the rotating member, causing the transferring member to take out the nucleic acid extracting element from the accommodation chamber and to transfer the nucleic acid extracting element into the reactor, causing the cylindrical member to remove the nucleic acid extracting element from the transferring member and accommodating the nucleic acid extracting element in the reactor, and causing the rotating member to attach the cap to the reactor.

When employing the nucleic acid amplification container including a plurality of reagent wells that store therein a plurality of reagents necessary for amplification of the target nucleic acid, the transferring member may be a nozzle used for dispensing or mixing the plurality of reagents in the nucleic acid amplification container.

The nozzle may be configured to aspire and discharge a liquid with a chip mounted thereon, and to take out the nucleic acid extracting element from the accommodation chamber when the chip is not mounted. More specifically, the chip is mounted on the nozzle when a tip portion thereof is fitted to the chip, and the nozzle takes out the nucleic acid extracting element from the accommodation chamber when the tip portion is fitted to a recessed portion provided on the nucleic acid extracting element.

In a preferred embodiment, the nucleic acid analyzing apparatus further includes a cylindrical member that encloses the nozzle, and is relatively movable in a vertical direction with respect to the nozzle. The cylindrical member removes the chip or the nucleic acid extracting element fitted to the tip portion of the nozzle when moved downward with respect thereto. In this case, it is preferable that the nucleic acid extracting element includes a projection that interferes with the cylindrical member when the nucleic acid extracting element is removed from the nozzle. It is preferable to mount an O-ring on the tip portion of the nozzle, at the position to be fitted to the chip or the nucleic acid extracting element.

In the present invention, the term “specimen” represents a concept including animal-derived biological specimen (for example whole blood, blood serum, blood plasma, urine, saliva, or fluid) as well as biological specimen originating from other than animals, and the term “nucleic acid” refers to DNA or RNA, and represents a concept including double-strand DNA, single-strand DNA, plasmid DNA, genome DNA, cDNA, foreign parasite (virus, bacteria, fungus or the like)-derived RNA, and endogenous RNA.

### BRIEF DESCRIPTION OF THE DRAWINGS

FIG. 1 is a perspective view showing an entirety of a nucleic acid analyzing apparatus for explaining an example thereof;

FIG. 2 is a plan view showing an internal structure of the nucleic acid analyzing apparatus shown in FIG. 1;

FIG. 3 is a cross-sectional view taken along the line III-III in FIG. 2;

FIG. 4 is a cross-sectional view taken along the line IV-IV in FIG. 2;

FIG. 5 is a perspective view showing an entirety of a nucleic acid purification cartridge;

FIG. 6 is a cross-sectional view taken along the line VI-VI in FIG. 5;

FIG. 7A is a perspective view showing an entirety of a nucleic acid extracting element in the nucleic acid purification cartridge, and FIG. 7B is a cross-sectional view of the nucleic acid extracting element;

FIG. 8 is a perspective view showing an entirety of a nucleic acid amplification cartridge;

FIG. 9 is a cross-sectional view taken along the line IX-IX in FIG. 8;

FIG. 10 is a fragmentary cross-sectional view for explaining a cleaning operation of a solid matrix;

FIGS. 11A and 11B are fragmentary cross-sectional views showing a cap removal operation from the nucleic acid amplification cartridge;

FIG. 12 is a fragmentary cross-sectional view showing an operation of taking out the nucleic acid extracting element utilizing the cap;

FIG. 13A is a fragmentary cross-sectional view showing an operation of accommodating the nucleic acid extracting element in a reactor in the nucleic acid amplification cartridge, and FIG. 13B is a fragmentary cross-sectional view showing an operation of removing the cap from the reactor;

FIG. 14 is a cross-sectional view taken along the line XIV-XIV in FIG. 13B;

FIG. 15 is a cross-sectional view corresponding to a cross-section taken along the line XV-XV in FIG. 2, for explaining a temperature control mechanism and measurement mechanism;

FIG. 16 is a plan view showing an internal structure of the nucleic acid analyzing apparatus for explaining an example thereof;

FIG. 17 is a cross-sectional view taken along the line XVII-XVII in FIG. 16;

FIG. 18 is a cross-sectional view taken along the line XVII-XVIII in FIG. 16;

FIG. 19 is a perspective view showing an entirety of a nucleic acid purification cartridge;

FIG. 20A is a perspective view showing a nucleic acid extracting element in the nucleic acid purification cartridge, FIG. 20B is a plan view thereof, and FIG. 20C is a cross-sectional view taken along the line XXc-XXc in FIG. 20A;

FIG. 21 is a cross-sectional view of a nucleic acid purification cartridge container corresponding to a cross-section taken along the line XV-XV in FIG. 19;

FIG. 22 is a fragmentary cross-sectional view showing an operation of taking out the nucleic acid extracting element from an accommodation chamber in the container;

FIG. 23 is a perspective view showing an entirety of a nucleic acid amplification cartridge;

FIG. 24A is a cross-sectional view taken along the line XXIVa-XXIVa in FIG. 23, and FIG. 24B is a cross-sectional view showing a state where the cap is removed in FIG. 24A;

FIGS. 25A and 25B are fragmentary front views for explaining an operation of attaching a chip to a nozzle;

FIGS. 26A and 26B are fragmentary front views for explaining an operation of attaching the nucleic acid extracting element to the nozzle;

FIGS. 27A to 27C are fragmentary front views for explaining an operation of removing the chip from the nozzle;

FIGS. 28A to 28C are fragmentary front views for explaining an operation of removing the nucleic acid extracting element from the nozzle;

FIGS. 29A and 29B are fragmentary cross-sectional views showing an operation of inserting a rotating member into the cap of the nucleic acid amplification cartridge;

FIG. 30 is a fragmentary cross-sectional view for explaining an operation of removing the cap from the nucleic acid amplification cartridge;

FIG. 31 is a fragmentary cross-sectional view showing an operation of accommodating the nucleic acid extracting element in the reactor in the nucleic acid amplification cartridge;

FIG. 32 is a fragmentary cross-sectional view for explaining an operation of reattaching the cap of the nucleic acid amplification cartridge;

FIG. 33 is a cross-sectional view corresponding to a cross-section taken along the line XXXIII-XXXIII in FIG. 16, for explaining the measurement mechanism;

FIG. 34 is a line graph showing measurement result of fluorescence intensity from Working Example 1 (PCR process), in which the horizontal axis represents a temperature, and the vertical axis a derivative value of the fluorescence intensity;

FIG. 35 is a line graph showing measurement result of fluorescence intensity from Working Example 2 (ICAN process), in which the horizontal axis represents the number of cycles, and the vertical axis the fluorescence intensity; and

FIG. 36 is a line graph showing measurement result of fluorescence intensity from Working Example 3 (LAMP process), in which the horizontal axis represents the number of cycles, and the vertical axis the fluorescence intensity.

### BEST MODE FOR CARRYING OUT THE INVENTION

Referring to the accompanying drawings, the present invention will be described below based on a first and a second embodiments.

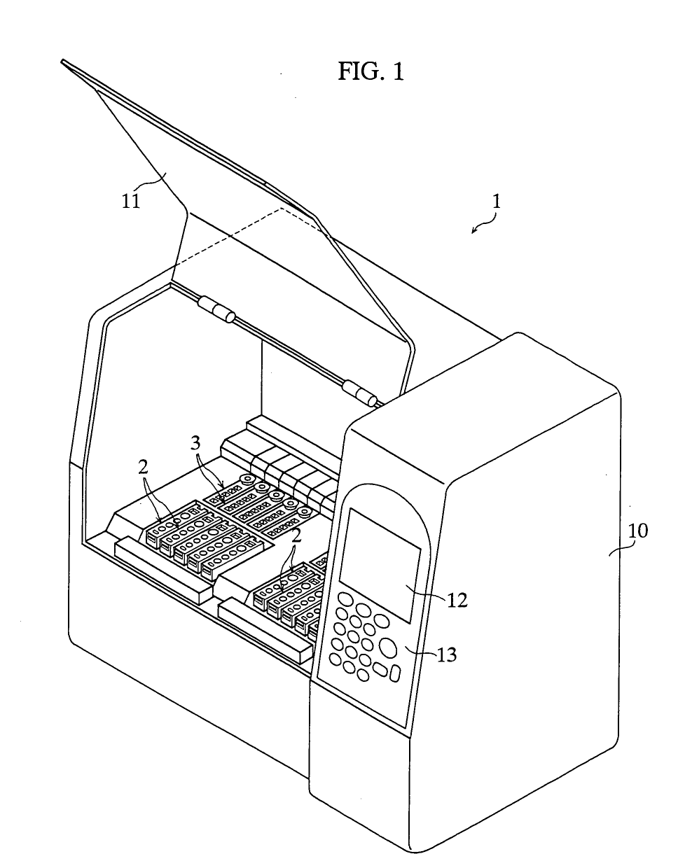

First, reference is made to FIGS. 1 through 15 illustrating the first embodiment of the present invention.

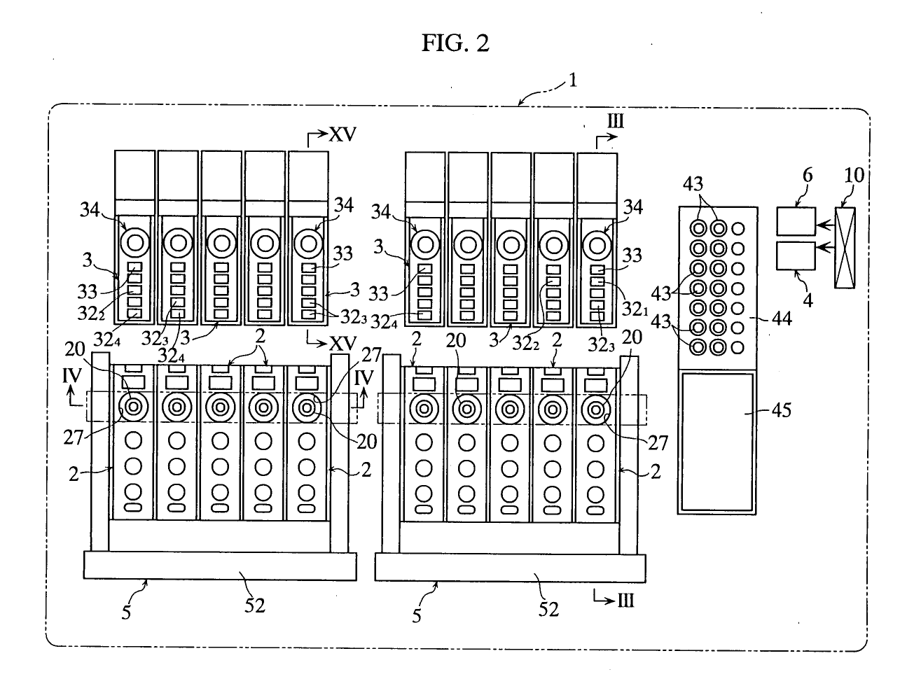

A nucleic acid analyzing apparatus 1 shown in FIGS. 1 to 4 is configured to automatically execute purification of nucleic acid in a specimen, amplification of the extracted nucleic acid, and analysis of the amplified nucleic acid and includes, as shown in FIGS. 1 and 2, a plurality of nucleic acid purification cartridges 2 and the same number of nucleic acid amplification cartridges 3, attached inside a casing 10.

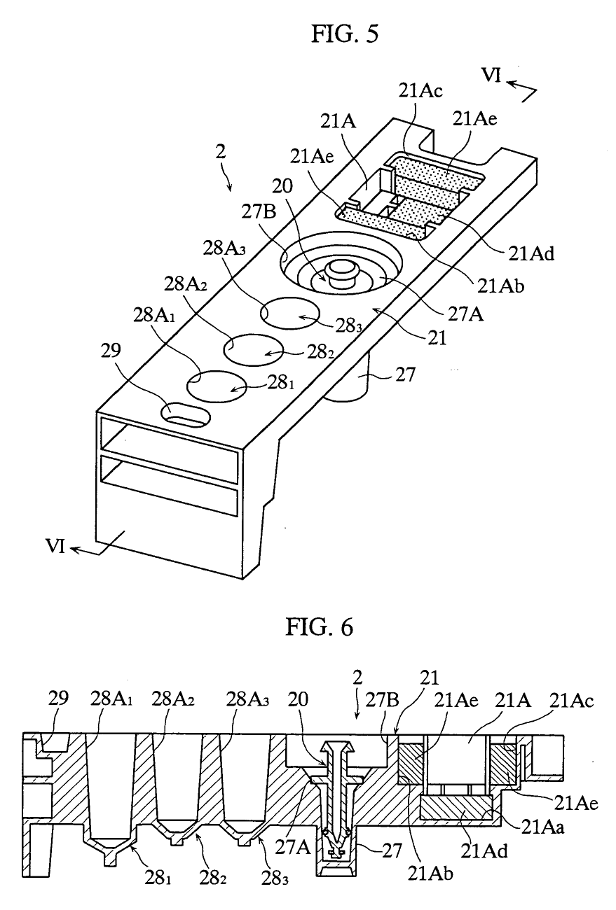

As shown in FIGS. 5 and 6, the nucleic acid purification cartridge 2 serves to enable the automatic purification of the nucleic acid in the nucleic acid analyzing apparatus 1, and includes a nucleic acid extracting element 20 and a cartridge main body 21.

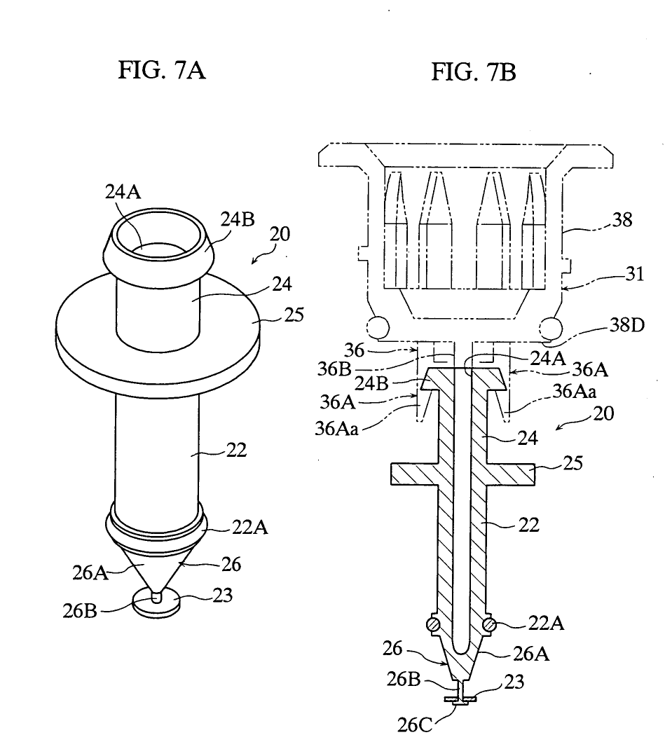

The nucleic acid extracting element 20, which is utilized for extracting the nucleic acid from the specimen, is accommodated in an accommodation chamber 27 of the cartridge main body 21 to be subsequently described. As explicitly shown in FIGS. 7A and 7B, the nucleic acid extracting element 20 includes a retaining member 22 and a solid matrix 23.

The retaining member 22 includes a cylindrical portion 24, a flange 25, and a retaining portion 26, and an entirety thereof is formed by, for example, a resin molding process.

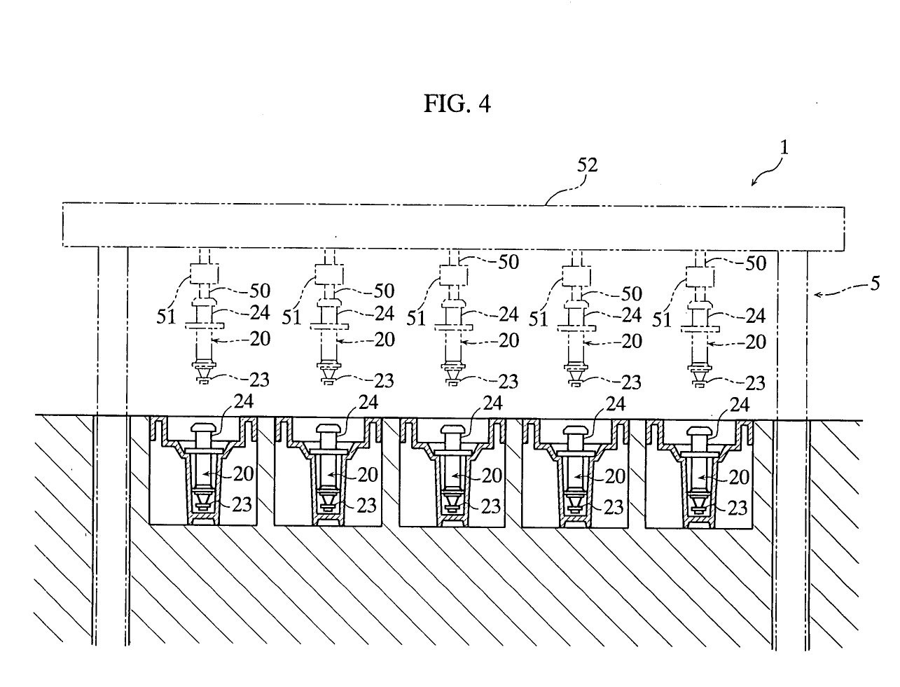

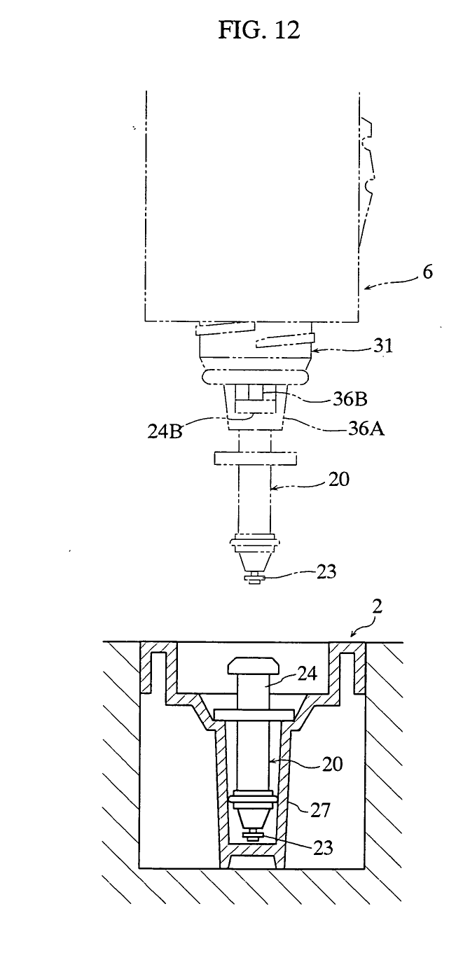

The cylindrical portion 24 is utilized when moving the nucleic acid extracting element 20 (refer to FIGS. 4 and 12), and includes a recessed portion 24A and an engaging head 24B. The recessed portion 24A is to be fitted to an insertion pin 50 of a nucleic acid purification mechanism 5 or a pin 36B of a cap 31 of the nucleic acid amplification cartridge 3, which will be subsequently described (refer to FIGS. 4 and 12). The engaging head 24B is to be fitted to an engaging pawl 36A of the cap 31 of the nucleic acid amplification cartridge 3 to be subsequently described, and is projecting in a radial direction.

The flange 25 is to be fitted to a stepped portion 27A of a accommodation chamber 27 when the nucleic acid extracting element 20 is accommodated in the accommodation chamber 27 of the nucleic acid purification cartridge 2 to be described later, and is of a ring shape radially projecting outward (refer to FIG. 12).

The retaining portion 26 serves to retain the solid matrix 23, and includes a tapered portion 26A, a pin-shaped portion 26B and a stopper piece 26C. The tapered portion 26A causes cleaning liquid remaining on the retaining portion 26 to flow downward. The pin-shaped portion 26B is to be pierced through the solid matrix 23. The stopper piece 26C serves to prevent the solid matrix 23 from coming off from the pin-shaped portion 26B (retaining portion 26), after the pin-shaped portion 26B is pierced through the solid matrix 23.

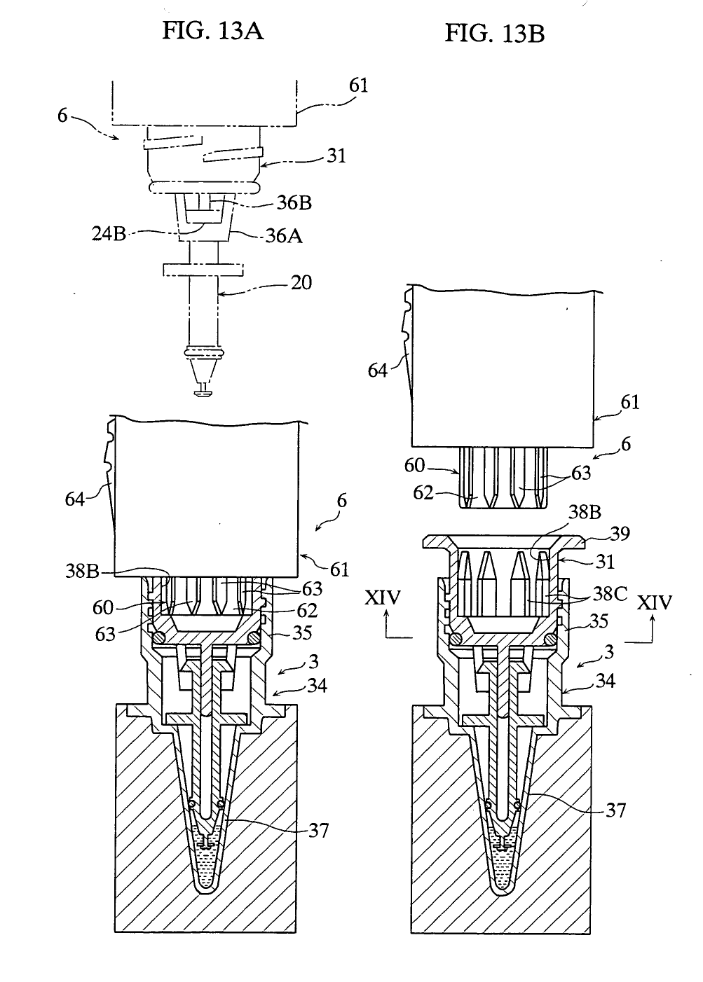

The retaining member 22 is provided with an O-ring 22A fixed to a position slightly above the retaining portion 26. As explicitly shown in FIG. 13B, the O-ring 22A serves to achieve close contact between the nucleic acid extracting element 20 and an inner surface of a reactor or reaction vessel 34, when the nucleic acid extracting element 20 is accommodated in the reactor 34 of the nucleic acid amplification cartridge 3. Accordingly, when the nucleic acid extracting element 20 is accommodated in the reactor 34, a sealed space is defined below the position where the O-ring 22A is in close contact with the reactor 34. Thus, since the O-ring 22A is located above the retaining portion 26, the solid matrix 23 is accommodated in the sealed space.

The solid matrix 23 serves to carry the nucleic acid contained in the specimen, and is for example formed of a filter paper with a reagent for extracting the nucleic acid provided thereon. The solid matrix 23 is of a disk shape. Thus, the solid matrix 23 is retained to be orthogonal to a vertical axis of the retaining member 22, i.e. in a horizontal or generally horizontal orientation, while being engaged with the pin-shaped portion 26B.

Here, examples of the reagents for extracting the nucleic acid include a combination of a weak base, a chelate reagent, a negative ion surfactant or a negative ion detergent and a uric acid or a urate, or a combination of a nucleic acid adsorbing carrier and an adsorption promoter. Various known nucleic acid adsorbing carriers are available, among which silica beads are typically employed. Such materials that destroy a cell membrane or modify a protein in the specimen to thereby encourage the bonding of the nucleic acid and the nucleic acid adsorbing carrier may be employed as the adsorption promoter, for example a chaotropic substance (such as a guanidine thiocyanate or guanidinate). Materials of the solid matrix are not limited to the foregoing examples, as long as the material efficiently causes the nucleic acid in the specimen to adsorb thereto.

The nucleic acid extracting element 20 can hold the solid matrix 23 at a position spaced from a bottom portion of the reactor 34 of the nucleic acid amplification cartridge 3, which will be described later, when accommodated therein. Such arrangement prevents interference of the solid matrix 23 with a photometric path of a photometric mechanism 8 to be subsequently described, thereby upgrading accuracy in photometry. Since the solid matrix 23 does not interfere with the photometric path, the solid matrix 23 may be formed in larger dimensions. This enables the solid matrix 23 to carry a greater amount of nucleic acid, thereby improving efficiency in amplifying the nucleic acid, as well as accuracy in analysis.

As shown in FIGS. 5 and 6, the cartridge main body 21 includes the accommodation chamber 27, three cleaner wells 28₁ to 28₃, a specimen well 29 and a surplus liquid removal chamber 21A, and is integrally formed for example by a resin molding process.

The accommodation chamber 27 serves to accommodate the nucleic acid extracting element 20, and includes the stepped portion 27A to be engaged with the flange 25 of the nucleic acid extracting element 20. It is preferable to cover an upper opening 27B of the accommodation chamber 27 with a sealing material such as an aluminum foil, to prevent the nucleic acid extracting element 20 from escaping through the upper opening 27B before the use of the nucleic acid purification cartridge 2. The sealing material may be stripped by the user or automatically stripped by the nucleic acid analyzing apparatus 1, when using the nucleic acid purification cartridge 2.

The cleaner wells 28₁ to 28₃ serve to store cleaning liquid which removes a foreign substance from the solid matrix 23 after causing the solid matrix 23 to carry the nucleic acid. Although it is preferable to load the cleaner wells 28₁ to 28₃ with the cleaning liquid in advance when preparing the nucleic acid purification cartridge 2, the cleaning liquid loaded in the nucleic acid analyzing apparatus 1 may be dispensed to the cleaner wells 28₁ to 28₃ when executing the analysis. As the cleaning liquid, such materials that barely elutes the nucleic acid from the solid matrix 23 and restrains bonding of the foreign substance may be employed, for example a guanidinate or ethanol. The three cleaner wells 28₁ to 28₃ may contain the same cleaning liquid, or different types of cleaning liquid from one another.

In the case of loading the cleaner wells 28₁ to 28₃ with the cleaning liquid in advance, it is necessary to cover upper openings 28A₁ to 28A₃ of the cleaner wells 28₁ to 28₃ with a sealing material such as an aluminum foil. In this case, the sealing material may individually cover the respective upper openings 28A₁ to 28A₃ Of the cleaner wells 28₁ to 28₃, or collectively cover the three upper openings 28A₁ to 28A₃ of the cleaner wells 28₁ to 28₃ or the three upper openings 28A₁ to 28A₃ of the cleaner wells 28₁ to 28₃ and the upper opening 27B of the accommodation chamber 27.

The specimen well 29 serves to store therein the specimen which is the object to be analyzed (object from which the nucleic acid is to be extracted). The specimen may be loaded in the specimen well 29 before setting the nucleic acid purification cartridge 2 in the nucleic acid analyzing apparatus 1, or after setting the nucleic acid purification cartridge 2 in the nucleic acid analyzing apparatus 1. In the latter case, it is preferable to configure the nucleic acid analyzing apparatus 1 to automatically dispense the specimen into the specimen well 29. Suitable examples of the specimen include whole blood, blood serum, blood plasma, urine, saliva, or fluid.

The surplus liquid removal chamber 21A serves to remove the surplus cleaning liquid remaining on the nucleic acid extracting element 20, the solid matrix 23, and the retaining portion 26 of the retaining member 22, after cleaning the solid matrix 23 of the nucleic acid extracting element 20. The surplus liquid removal chamber 21A is provided with water-absorbent materials 21Ad, 21Ae fixed in close contact with a bottom wall 21Aa and a front and rear wall 21Ab, 21Ac. The water-absorbent materials 21Ad, 21Ae are constituted of a porous material such as a foam resin or a cloth, to absorb and remove the surplus cleaning liquid from the nucleic acid extracting element 20, upon being contacted thereby.

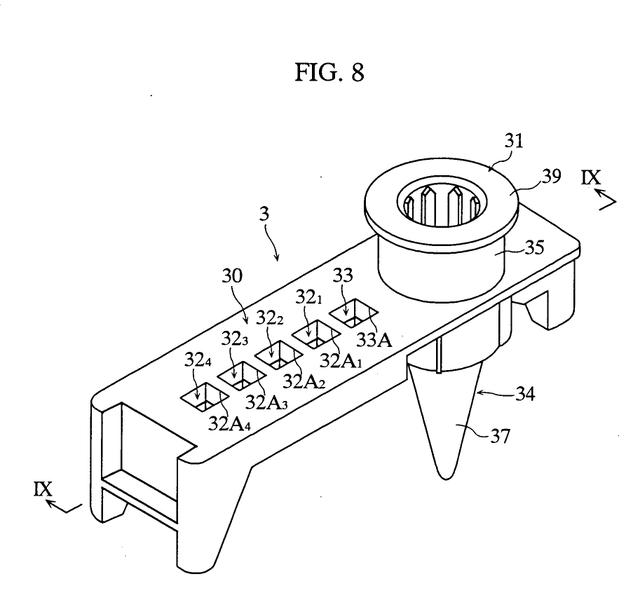

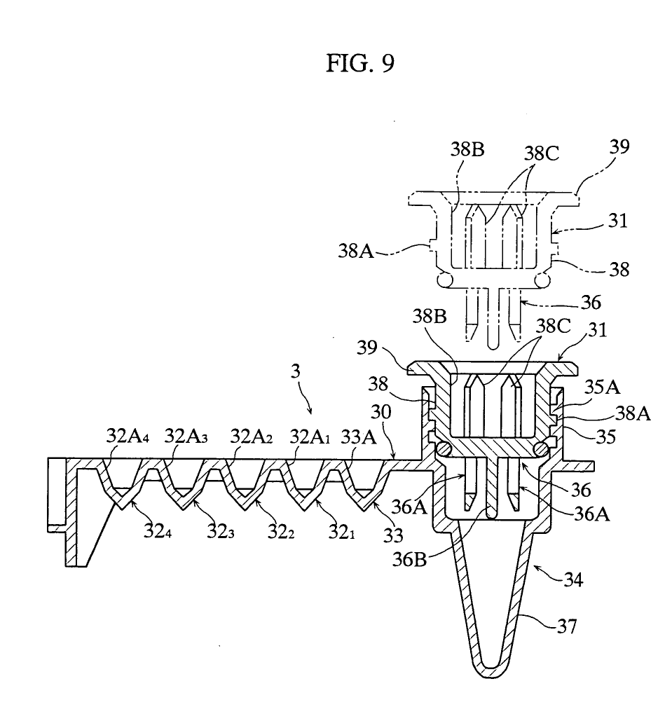

As shown in FIGS. 8 and 9, the nucleic acid amplification cartridge 3 serves to enable the nucleic acid analyzing apparatus 1 to execute the automatic amplification and measurement of the nucleic acid, and includes a cartridge main body 30 and the cap 31.

The cartridge main body 30 includes four reagent wells 32₁ to 32₄, a mixing well 33, and a reactor 34, and is integrally formed for example by a resin molding process.

The reagent wells 32₁ to 32₄ serve to retain the reagent necessary for the amplification and measurement of the nucleic acid in a form of a solution or suspension. Here, the types of the reagent to be loaded in the reagent wells 32₁ to 32₄ are selected in accordance with the amplification method or measurement method to be adopted. Applicable amplification methods include the Polymerase Chain Reaction (PCR) process, an Isothermal and Chimeric Primer-initiated Amplification of Nucleic acid (hereinafter, ICAN) process, a Loop-Mediated Isothermal Amplification (hereinafter, LAMP) process and a Nucleic acid Sequence Based Amplification (hereinafter, NASBA) process. When adopting the PCR process, at least two types of primers, dNTP, and DNA polymerase are employed as the reagent. When adopting the ICAN process, a chimera primer, DNA polymerase, and RNaseH are employed as the reagent. When adopting the LAMP process, at least one type of LAMP primer, dNTP, chain-substituted DNA syntethase, and a reverse transcriptase are used as the reagent. When adopting the NASBA process, at least two types of primers, dNTP, rNTP, reverse transcriptase, DNA polymerase, RNaseH, and RNA polymerase are employed as the reagent. Applicable measurement methods include fluorometry, luminescent measurement, radioactive measurement, and electrophoresis. In the nucleic acid analyzing apparatus 1, however, it will be assumed that the fluorometry is adopted. In this case, it is preferable to employ a fluorescent primer as the primer.

The mixing well 33 is utilized to mix two or more reagents retained in the reagent wells 32₁ to 32₄, before supplying the reagents to the reactor 34.

Although it is preferable to load the reagent wells 32₁ to 32₄ with the reagent in advance, the reagent loaded in the nucleic acid analyzing apparatus 1 may be dispensed to the reagent wells 32₁ to 32₄ when executing the analysis. In this case, it is necessary to cover upper openings 32A₁ to 32A₄ of the reagent wells 32₁ to 32₄ with a sealing material such as an aluminum foil, and the sealing material may individually cover the respective upper openings 32A₁ to 32A₄ of the reagent wells 32₁ to 32₄, or collectively cover the four upper openings 32A₁ to 32A₄ of the reagent wells 32₁ to 32₄ or the four upper openings 32A₁ to 32A₄ of the reagent wells 32₁ to 32₄ and an upper opening 33A of the mixing well 33.

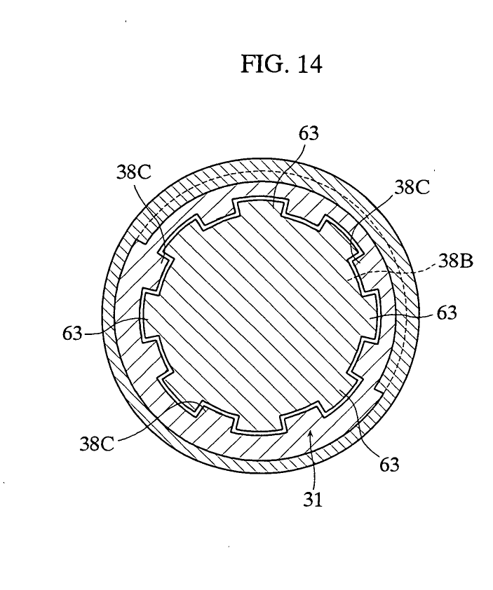

The reactor 34 serves to accommodate the mixed reagent and the nucleic acid extracting element 20, as well as to provide a location where the nucleic acid carried by the nucleic acid extracting element 20 and the mixed reagent prepared in the mixing well 33 are reacted (refer to FIGS. 13 and 14). The reactor 34 includes a cylindrical portion 35 and a reaction detecting portion 37.

The cylindrical portion 35 is where the cap 31 is to be mounted, and includes a female thread formed on an inner circumferential surface thereof.

The reaction detecting portion 37 provides a location where the amplification reaction of the nucleic acid takes place, as well as serves as a detecting container for executing the fluorometry. In other words, the reaction detecting portion 37 is the portion to be irradiated with a light emitted by a light emitter 80 of the photometric mechanism 8, which will be subsequently described (refer to FIG. 15).

The cap 31 is utilized to select whether the inside of the reaction detecting portion 37 is sealed, and is removably attachable to the reactor 34 (cylindrical portion 35). More specifically, the cap 31 is subjected to a rotational force, to select either being mounted on the cylindrical portion 35 or being completely separated from the cylindrical portion 35 (reactor 34). The cap 31 includes a cylindrical-shaped main body 38, a flange 39 and a holder 36.

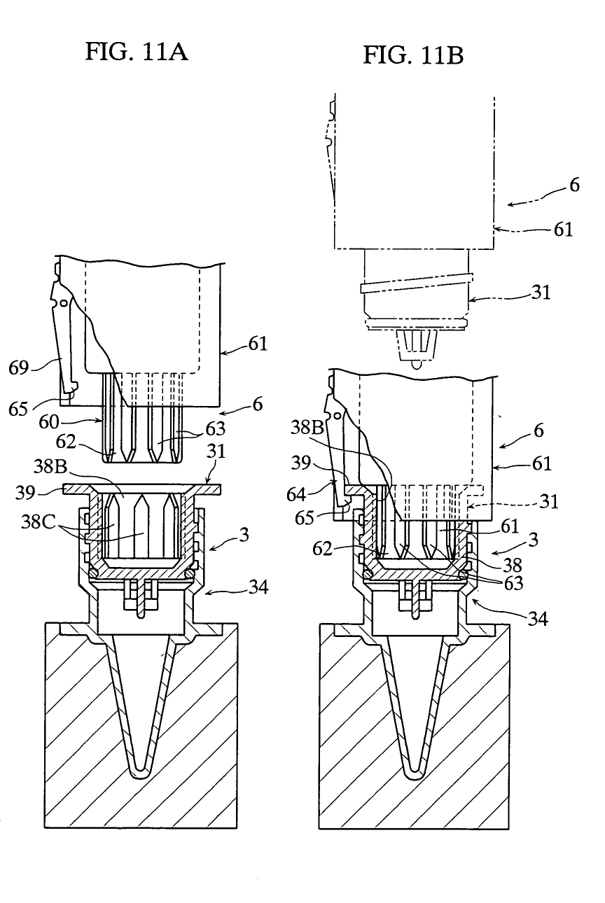

The main body 38 includes a male thread 38A to be screw-fitted to the female thread 35A of the cylindrical portion 35 of the reactor 34, and a recessed portion 38B in which a rotating member 60 (refer to FIG. 11B) of a cap attaching/removing mechanism 6, which will be described later, is to be inserted. The recessed portion 38B includes a plurality of ribs 38C formed on an inner circumferential surface thereof. The ribs 38C are oriented to vertically extend and circumferentially aligned at regular intervals. An upper end portion of each rib 38C is of a tapered shape, with a decreasing width toward the upper end.

The flange 39 is to be engaged with a pawl 64 of an enclosing member 61 of the cap attaching/removing mechanism 6 to be subsequently described, when the cap 31 removed from the reactor 34 is moved (refer to FIG. 11B). The flange 39 is of a ring shape radially projecting outward from an upper end portion of the main body 38.

As shown in FIG. 7B, the holder 36 serves to retain the nucleic acid extracting element 20 of the nucleic acid purification cartridge 2, and includes a pair of engaging pawls 36A and a pin 36B.

The pair of engaging pawls 36A is to be engaged with the engaging head 24B of the nucleic acid extracting element 20, and projecting downward from a bottom surface 38D of the main body 38. The engaging pawls 36A include a hook portion 36Aa at a tip portion thereof, and the hook portion 36Aa is swingable. Accordingly, the hook portions 36Aa of the pair of engaging pawls 36A can move toward or away from each other.

The pin 36B is to be inserted in the recessed portion 24A in the cylindrical portion of the nucleic acid extracting element 20, and projecting downward from the bottom surface 38D of the main body 38. The pin 36B serves as a guide when the cap 31 retains the nucleic acid extracting element 20, as well as suppresses a rattling motion of the nucleic acid extracting element 20 against the cap 31, after the cap 31 gets engaged with the nucleic acid extracting element 20.

Referring back to FIG. 1, the casing 10 of the nucleic acid analyzing apparatus 1 is provided with a lid 11, a display unit 12 and an operation panel 13. The lid 11 is utilized to select whether the inside of the casing 10 is exposed, such that the lid 11 is opened when the cartridges 2, 3 are introduced into or taken out from the casing 10, and is closed when executing the analysis of the nucleic acid or when the nucleic acid analyzing apparatus 1 is not in use. The display unit 12 serves to display an analysis result and so forth, and includes for example an LCD. The operation panel 13 is manipulated for setting various parameters, starting the analysis and so forth.

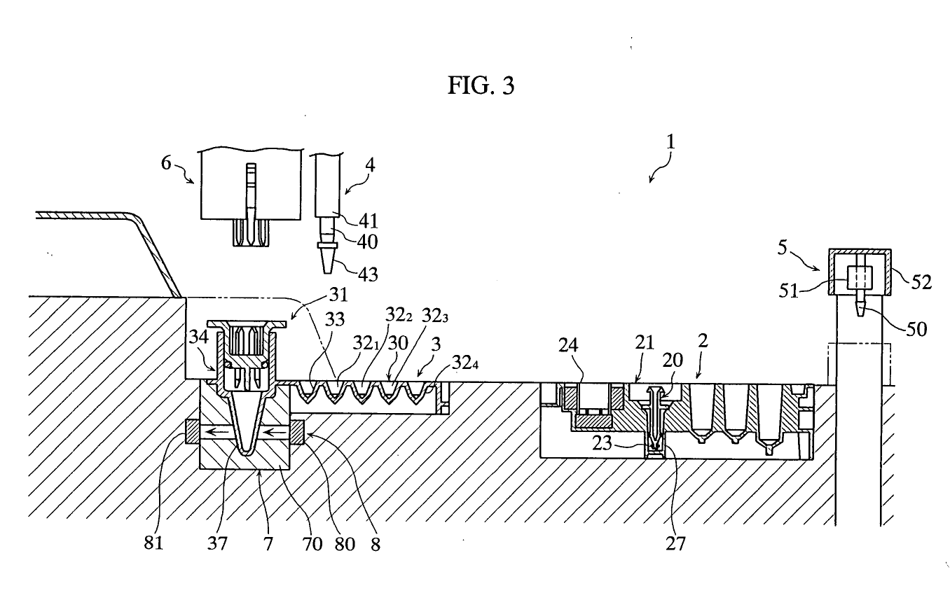

As shown in FIGS. 2 and 3, the casing 10 contains a pipet device 4, the nucleic acid purification mechanism 5, the cap attaching/removing mechanism 6, a temperature control mechanism 7, and the photometric mechanism 8.

The pipet device 4 primarily serves to prepare a mixed solution in the nucleic acid amplification cartridge 3, and includes a nozzle 40. The pipet device 4 is utilized to supply the specimen or the cleaning liquid, as the case may be, to the nucleic acid purification cartridge 2.

The nozzle 40 is connected to a pump (not shown) to aspire and discharge a liquid, and configured to select either applying a suction force or a discharging force into or out of the inside of the nozzle 40. The nozzle 40 is movable in both vertical and horizontal directions by a driving mechanism (not shown) such as a robot arm, and the movement of the nozzle 40 is controlled by a control unit 10 that includes a CPU or the like. The nozzle 40 can be moved to the reagent well 32₁ to 32₄, the mixing well 33, and the reactor 34 of the nucleic acid amplification cartridge 3, and to the accommodation chamber 27 of the nucleic acid purification cartridge 2. When the mixed specimen is prepared or when the mixed specimen is dispensed to the reactor 34 (reaction detecting portion 37), a chip 43 is attached to a tip portion 42 of the nozzle 40, as shown in FIG. 3. The chip 43 is, as shown in FIG. 2, placed on a rack 44 at a position adjacent to a standby position of the nozzle 40 (pipet device 4). At the location adjacent to the rack 44, a waste box 45 is provided, in which the used chip 43 is disposed.

As shown in FIGS. 2 to 4 and 10, the nucleic acid purification mechanism 5 serves to control the movement of the nucleic acid extracting element 20, when extracting the nucleic acid in the specimen with the nucleic acid extracting element 20 of the nucleic acid purification cartridge 2. The nucleic acid purification mechanism 5 includes a plurality of insertion pins 50, a cylindrical element 51 and a support frame 52.

The insertion pins 50 are to be fitted to the cylindrical portion 24 of the nucleic acid extracting element 20, and supported by a support frame 52 to move together.

The cylindrical element 51 serves to remove the nucleic acid extracting element 20 attached to the insertion pin 50, and encloses the insertion pin 50 such that the cylindrical element 51 is movable independently from the insertion pin 50, in a vertical direction. In other words, the cylindrical element 51 is located above the nucleic acid extracting element 20 (standby position) except when the nucleic acid extracting element 20 is removed from the insertion pin 50, and is relatively moved downward with respect to the insertion pin 50 when the nucleic acid extracting element 20 is removed from the insertion pin 50.

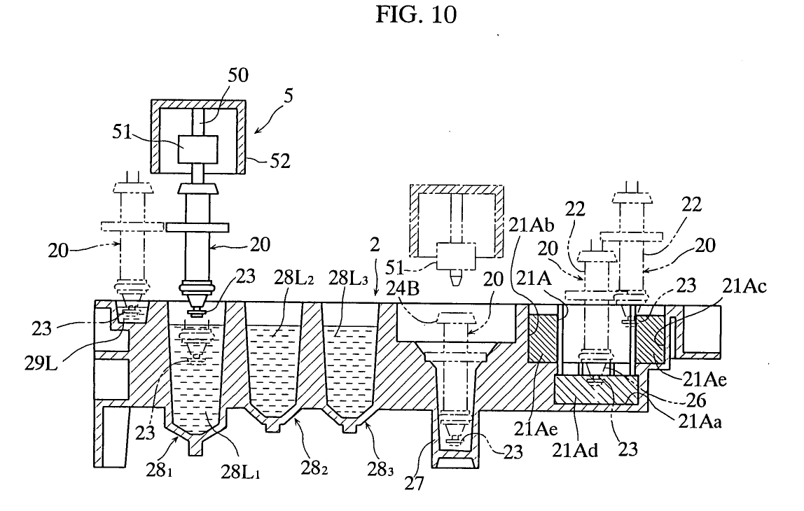

The support frame 52 supports the plurality of insertion pins 50 aligned at regular intervals along a direction in which the plurality of nucleic acid purification cartridges 2 are aligned, and serves as a medium that moves the insertion pins 50. The support frame 52 is installed to be moved by a driving mechanism (not shown) in a vertical and horizontal direction, and the movement thereof is controlled, for example, by the control unit 10 shown in FIG. 2. Such structure allows the plurality of insertion pins 50, and hence the nucleic acid extracting element 20 attached thereto, to move in a vertical and horizontal direction with the support frame 52. Accordingly, the plurality of nucleic acid extracting elements 20 can be collectively moved to impregnate each solid matrix 23 with the specimen, clean each solid matrix 23 and remove the surplus liquid at a time (refer to FIG. 10).

As shown in FIGS. 11 and 13, cap attaching/removing mechanism 6 serves to remove the cap 31 from the reactor 34 of the nucleic acid amplification cartridge 3 and to attach the cap 31 to the reactor 34, and includes a rotating member 60 and the enclosing member 61. The rotating member 60 and the enclosing member 61 are movable by a driving mechanism (not shown) in a vertical and horizontal direction, and the movement thereof is controlled by the control unit 10 (refer to FIG. 2).

The rotating member 60 serves to apply a rotational force to the cap 31 of the nucleic acid amplification cartridge 3 and to retain and move the cap 31, and includes a generally column-shaped tip portion 62. The tip portion 62 of the rotating member 60 includes a plurality of ribs 63. The plurality of ribs 63 is formed to vertically extend and aligned at regular intervals circumferentially of the tip portion 62, and a lower end portion of each rib 63 is of a tapered shape with a decreasing width toward the lower end. The ribs 63 are to be engaged with the plurality of ribs 38 of the cap 31 as shown in FIG. 14, so that when the tip portion 62 is inserted into the recessed portion 38B of the cap 31 each of the ribs 63 is located between the adjacent ones of the ribs 38 on the recessed portion 38B.

Under such structure, when the tip portion 62 of the rotating member 60 is rotated, the ribs 63 on the tip portion 61 and the ribs 38 on the recessed portion 38B interfere with one another, thereby inhibiting free rotation of the tip portion 62 inside the recessed portion 38B of the cap 31 and thus properly exerting the rotational force of the rotating member 60 to the cap 31. Also, the upper end portion of the plurality of ribs on the recessed portion 38B are of the tapered shape with a reducing width toward the upper end, while the lower end portion of the plurality of ribs 63 on the tip portion 61 of the rotating member 60 are of the tapered shape with a reducing width toward the lower end. Such configuration allows easily and securely inserting the tip portion 61 of the rotating member 60 into the recessed portion 38B of the cap 31.

The enclosing member 61 encloses the rotating member 60, and is of a cylindrical shape. The enclosing member 61 includes a pawl 64 to be engaged with the flange 39. The pawl 64 includes a hook portion formed at a tip portion 64 thereof, and the hook portion 65 is swingably disposed. The pawl 64 is engaged with the flange 39 of the cap 31, when the tip portion 62 of the rotating member 60 is inserted into the recessed portion 38B of the cap 31. Accordingly, the cap 31 is coupled with the rotating member 60, so that moving the rotating member 60 and the enclosing member 61 enables moving the cap 31. The pawl 62 is configured to be automatically disengaged from the flange 39 of the cap 31, when the rotating member 60 reattaches the cap 31 to the reactor 34.

As shown in FIG. 15, the temperature control mechanism 7 controls a temperature of a heat block 70, to thereby control a temperature of a liquid retained by the reaction detecting portion 37 of the nucleic acid amplification cartridge 3. The temperature of the heat block 70 is monitored by a sensor (not shown), so that the temperature of the heat block 70 is used for a feedback control based on the monitoring result from the temperature sensor. The heat block 70 includes a recessed portion 71 of a shape corresponding to the outer shape of the reaction detecting portion 37 of the nucleic acid amplification cartridge 3. Such configuration enables selectively and efficiently controlling the temperature of the reactor 34 with the heat block 7. The heat block 70 further includes linear through holes 72, 73 communicating with the recessed portion 71. The through hole 72 guides a light emitted by the light emitter 80 of the photometric mechanism 8, which will be subsequently described, to the reaction detecting portion 37 of the reactor 34, and the through hole 73 guides the light that has passed through the reaction detecting portion 37 to a photodetector 81.

The photometric mechanism 8 includes the light emitter 80 and the photodetector 81. The light emitter 80 irradiates the reaction detecting portion 37 with an exciting light via the through hole 72. The photodetector 81 receives via the through hole 73 a fluorescence generated when the reaction detecting portion 37 is irradiated with the exciting light. The photometric mechanism 8 causes the light emitter 80 to continuously emit the exciting light, while continuously monitoring the amount of the fluorescence by the photodetector 81, thereby recognizing the progress of the amplification of the nucleic acid at real time.

Now, an operation of the nucleic acid analyzing apparatus 1 will be described.

When analyzing the nucleic acid with the nucleic acid analyzing apparatus 1, the nucleic acid purification cartridge 2 and the nucleic acid amplification cartridge 3 are first installed in the nucleic acid analyzing apparatus 1, as shown in FIGS. 1 to 4. The number of cartridges 2, 3 to be installed may be any number as long as the number of nucleic acid purification cartridges 2 and that of nucleic acid amplification cartridges 3 are the same. In the subsequent description, it will be assumed that the cleaner well 28₁ to 28₃ of the nucleic acid purification cartridge 2 are loaded in advance with the cleaning liquid, and that the specimen well 29 is loaded in advance with the specimen before the nucleic acid purification cartridge 2 is installed in the nucleic acid analyzing apparatus 1.

Then conditions according to the number of cartridges 2, 3 installed in the nucleic acid analyzing apparatus 1 and the types of the cartridges 2, 3 (purification method, amplification method, measurement method) are set by manipulating the operation panel 13, confirming the settings on the display unit 12 provided on the nucleic acid analyzing apparatus 1. When the setting is completed, the nucleic acid analyzing apparatus 1 automatically executes the purification, amplification and measurement of the nucleic acid.

As shown in FIG. 4, the purification of the nucleic acid is executed through moving the nucleic acid extracting element 20 in the nucleic acid purification cartridge 2, by the nucleic acid purification mechanism 5.

More specifically, firstly the insertion pins 50 of the nucleic acid purification mechanism 5 are brought to a position right above the accommodation chamber 27 in the container 21 of the nucleic acid purification cartridge 2, and the support frame 52 is driven to move the insertion pins 50 downward, and then upward. When the insertion pins 50 are moved downward, each insertion pins 50 is fitted to the cylindrical portion 24 of the nucleic acid extracting element 20, so that the plurality of nucleic acid extracting elements 20 is coupled to the nucleic acid purification mechanism 5, and when the insertion pins 50 are moved upward the nucleic acid extracting elements 20 are elevated by the nucleic acid purification mechanism 5.

Referring then to FIG. 10, the insertion pins 50 are moved together with the support frame 52, and the solid matrix 23 of the nucleic acid extracting element 20 is dipped in the specimen 29L retained in the specimen well 29 in the nucleic acid purification cartridge 2. This causes the solid matrix 23 to carry the nucleic acid in the specimen 29L.

Each solid matrix 23 is then sequentially dipped in the cleaning liquid 28L₁ to 28L₃ respectively retained in the three cleaner wells 28₁ to 28₃. More specifically, the solid matrix 23 is cleaned through repeatedly moving the solid matrix 23 up and down by the nucleic acid purification mechanism 5. In this process, the nucleic acid purification mechanism 5 is controlled such that the solid matrix 23 is repeatedly dipped completely in the cleaning liquid 28L₁ to 28L₃ and lifted up to a position above the surface of the cleaning liquid 28L₁ to 28L₃.

Through such cleaning process, the solid matrix 23 is caused to strike the liquid surface when moved downward from the position above the liquid surface to be dipped in the cleaning liquid 28L₁ to 28L₃. At this moment, since the solid matrix 23 is retained in a horizontal or generally horizontal orientation, a large load is applied to the solid matrix 23. On the other hand, when the solid matrix 23 is moved inside the cleaning liquid 28L₁ to 28L₃, a large transfer resistance is applied to the solid matrix 23 since the solid matrix 23 is retained in a horizontal or generally horizontal orientation, which acts as a load that generates convection of the cleaning liquid. Such effects contribute to efficiently removing a foreign substance from the solid matrix 23. Accordingly, disturbance by the foreign substance against the amplification of the nucleic acid is effectively prevented in the subsequent amplification process of the nucleic acid, and the analysis of the nucleic acid can be accurately executed. Such effects can also be obtained when the solid matrix 23 is moved in an inclined orientation with respect to a vertical axis, not only when the solid matrix 23 is moved in a horizontal orientation.

Finally, a tip portion of the nucleic acid extracting element 20 is brought into contact with the water-absorbent materials 21Ad, 21Ae provided in the surplus liquid removal chamber 21A. Since the water-absorbent material 21Ad is disposed to cover the bottom wall 21Aa and the front and rear wall 21Ab, 21Ac of the surplus liquid removal chamber 21A, causing the tip portion of the nucleic acid extracting element 20 to contact all the portions of such water-absorbent material 21Ad allows efficiently removing the surplus cleaning liquid from the tip portion of the nucleic acid extracting element 20, in particular from the solid matrix 23 and the retaining portion 26 of the retaining member 22. As a result, disturbance by the foreign substance contained in the cleaning liquid against the amplification of the nucleic acid is effectively prevented, when subsequently amplifying the nucleic acid with the nucleic acid extracting element 20.

Once the cleaning process is completed, the solid matrix 23 may be blow-dried while still retained by the nucleic acid purification mechanism 5. After the cleaning process (or the blow-drying process as the case may be) of the solid matrix 23 is completed, the nucleic acid extracting element 20 is removed from the insertion pin 50, and accommodated back in the accommodation chamber 27 in the nucleic acid purification cartridge 2. The removal of the nucleic acid extracting element 20 from the insertion pin 50 is executed, as already stated, by downwardly moving the cylindrical element 51 of the nucleic acid purification mechanism 5, so that the cylindrical portion 51 interferes with the engaging head 24B.

Thus, since the nucleic acid purification cartridge 2 is configured to cause a solid substance (nucleic acid extracting element 20) to carry the nucleic acid, the solid matrix 23 can be easily moved inside the nucleic acid analyzing apparatus 1. From such viewpoint, the structure of the nucleic acid purification cartridge 2 is beneficial for automatically executing the analysis of the nucleic acid.

The nucleic acid amplification is executed through preparing the mixed reagent in the nucleic acid amplification cartridge 3, dispensing the mixed reagent to each reactor 34 of the nucleic acid amplification cartridge 3, and then accommodating the solid matrix 23 carrying the nucleic acid in the reactor with the retaining member 22. It is to be noted that once the mixed reagent and the solid matrix 23 are both accommodated in the reactor 34, the temperature of the heat block 70 (refer to FIG. 15) is controlled according to the adopted amplification method, thus to control the temperature of the reactor 34.

For the preparation of the mixed reagent, the chip 43 is attached to the tip portion 42 of the nozzle 40 of the pipet device 4, and a predetermined amount of the reagent retained in the reagent wells 32₁ to 32₄ of the nucleic acid amplification cartridge 3 is sequentially dispensed into the mixing well 33, and the pipet device 4 performs a pipetting operation to mix the dispensed solution (refer to FIG. 3).

The mixed solution is dispensed to the reactor 34 by the pipet device 4, with the cap 31 removed from the reactor 34 by the cap attaching/removing mechanism 6. As shown in FIGS. 11A and 11B, the cap 31 is removed by the cap attaching/removing mechanism 6 through inserting the tip portion 62 of the rotating member 60 of the cap attaching/removing mechanism 6 into the recessed portion 38B of the cap 31, and then rotating the rotating member 60 to move the cap 31 upward. When the rotating member 60 is inserted into the recessed portion 38B, the hook portion 65 of the pawl 64 of the enclosing member 61 is engaged with the flange 39 of the cap 31. Accordingly, the cap 31 removed from the reactor 34 can be moved with the rotating member 60 and the enclosing member 61. Thus in the nucleic acid analyzing apparatus 1 and the nucleic acid amplification cartridge 3, the configuration that allows easily and securely removing the cap 31 from the nucleic acid amplification cartridge 3 is achieved, to thereby attain the total automation of the nucleic acid amplification and the nucleic acid analysis.

Meanwhile, for the accommodation of the solid matrix 23 in the reactor 34, the cap attaching/removing mechanism 6 and the cap 31 of the nucleic acid amplification cartridge 3 are utilized. More specifically, the accommodation of the solid matrix 23 is, as shown in FIGS. 12 and 13, executed through a series of operations such as engaging the nucleic acid extracting element 20 with the cap 31 and reattaching the cap 31 to the reactor 34.

As shown in FIGS. 7B and 12, the nucleic acid extracting element 20 is engaged with the cap 31 by causing the cap attaching/removing mechanism 6 to locate the cap 31 at a position above the accommodation chamber 27 of the nucleic acid purification cartridge 2, and then to move the cap 31 downward. Through the process of moving the cap 31 downward, the pin 36B of the cap 31 is inserted into the recessed portion 24A in the cylindrical portion 24 of the nucleic acid extracting element 20. Accordingly, the positional relationship between the cap 31 and the cylindrical portion 24 of the nucleic acid extracting element 20 is delimited, so that the pair of engaging pawls 36A of the cap 31 is properly led to the position corresponding to the engaging head 24B of the cylindrical portion 24. Such movement causes the pair of engaging pawls 36A to be pressed from above against the engaging head 24B. As a result, the pair of engaging pawls 36A is displaced such that the respective hook portions 36Aa move away from each other. When the pair of engaging pawls 36A is moved farther downward, the pin 36B of the cap 31 is inserted deeper into the recessed portion 24A of the cylindrical portion 24, and also the hook portions 36Aa move toward each other upon reaching a position below the engaging head 24B. Consequently the pair of engaging pawls 36A gets engaged with the engaging head 24B, so that the nucleic acid extracting element 20 is retained by the cap 31. Such state is securely maintained because the pin 36B of the cap 31 is inserted into the recessed portion 24A of the cylindrical portion 24, and the nucleic acid extracting element 20 is prevented from rattling with respect to the cap 31.

As shown in FIG. 13, the cap 31 is reattached by rotating the rotating member 60 retaining the cap 31, with the cap 31 being positioned with the reactor 34. In other words, exerting the rotational force to the properly positioned cap 31 causes the cap 31 to be screw-fitted to the cylindrical portion 35 of the reactor 34. When the cap 31 is screw-fitted to the cylindrical portion 35, the pawl 64 of the enclosing member 61 is disengaged from the flange 39 of the cap 31. This permits the rotating member 60 and the enclosing member 61 to move independently from the cap 31. On the other hand, since the cap 31 retains the nucleic acid extracting element 20, the nucleic acid extracting element 20 is accommodated in the reactor 34. As already stated, the nucleic acid extracting element 20 is provided with the O-ring 22A at a position slightly above the retaining portion 26, and hence the solid matrix 23 on the nucleic acid extracting element 20 is fixed in a sealed space at a position spaced by a predetermined distance from the bottom portion of the reactor 34. Since the mixed reagent is already loaded in the reaction detecting portion 37, the entirety of the solid matrix 23 is dipped, in the reaction detecting portion 37. Therefore, the nucleic acid is eluted from the solid matrix 23, and the eluted nucleic acid is reacted with the reagent, thus to be amplified.

Thus, in the nucleic acid analyzing apparatus 1, the nucleic acid extracting element 20 in the accommodation chamber 27 can be transferred to and accommodated in the reactor 34, utilizing the cap attaching/removing mechanism 6, which is provided for attaching and removing the cap 31. Accordingly, the nucleic acid analyzing apparatus 1 eliminates the need to provide an independent mechanism that transfers the nucleic acid extracting element 20. Such arrangement prevents the apparatus from becoming complicated despite aiming at executing the purification and amplification of the nucleic acid with a single apparatus, thereby suppressing an increase in dimensions of the apparatus, thus offering another advantage of suppressing an increase in number of mechanisms to be controlled.

As shown in FIG. 15, the measurement of the nucleic acid is executed by the photometric mechanism 8, with the reactor 34 covered with a light-shielding member 9 disposed thereabove.

In the photometric mechanism 8, the light emitter 80 irradiates the reaction detecting portion 37 of the reactor 34 with the exciting light, and the photodetector 81 receives the fluorescence thereby generated on the reaction detecting portion 37. As already stated, since the solid matrix 23 is set at a position that prevents disturbance against the measurement by the photometric mechanism 8, the measurement of the nucleic acid can be accurately executed in the nucleic acid analyzing apparatus 1.

As described above, the nucleic acid analyzing apparatus 1 is capable of automatically analyzing the nucleic acid, simply by installing the set of the nucleic acid purification cartridge 2 and the nucleic acid amplification cartridge 3 configured as above. The nucleic acid purification cartridge 2 and the nucleic acid amplification cartridge 3 include various advantageous features that facilitate automatically executing the nucleic acid analysis. Accordingly, when employing the nucleic acid analyzing apparatus 1, the nucleic acid purification cartridge 2, and the nucleic acid amplification cartridge 3, installing the cartridges 2, 3 in the nucleic acid analyzing apparatus 1 is the only step that depends on the manual operation by the user, for executing the nucleic acid extraction and the nucleic acid amplification. Such structure, therefore, significantly alleviates the burden on the user when executing the nucleic acid analysis, and minimizes the degradation in measurement reproducibility due to a difference in skill among the users which may create fluctuation in collection efficiency of the nucleic acid.

The present invention is not limited to the example described based on the foregoing embodiment. For example, it is not mandatory to retain the solid matrix of the nucleic acid extracting element in a horizontal or generally horizontal orientation with respect to the vertical axis of the retaining member, to form the solid matrix in a disk shape, or to pierce the solid matrix with the retaining member for retaining the solid matrix.

Also, for causing the cap 31 to retain the nucleic acid extracting element 20, for example the pawl may be provided on the nucleic acid extracting element, and the cap 31 may include an engaging portion to be engaged with the pawl, or the cap 31 and the nucleic acid extracting element 20 may be engaged only by a fitting force. Further, when engaging the cap 31 with the nucleic acid extracting element 20, the guide mechanism (the pin 36B of the cap 31 and the recessed portion 24A of the nucleic acid extracting element 20 in this embodiment) may be omitted, or may be constituted of a recessed portion formed on the cap 31 and a pin provided on the nucleic acid extracting element 30.

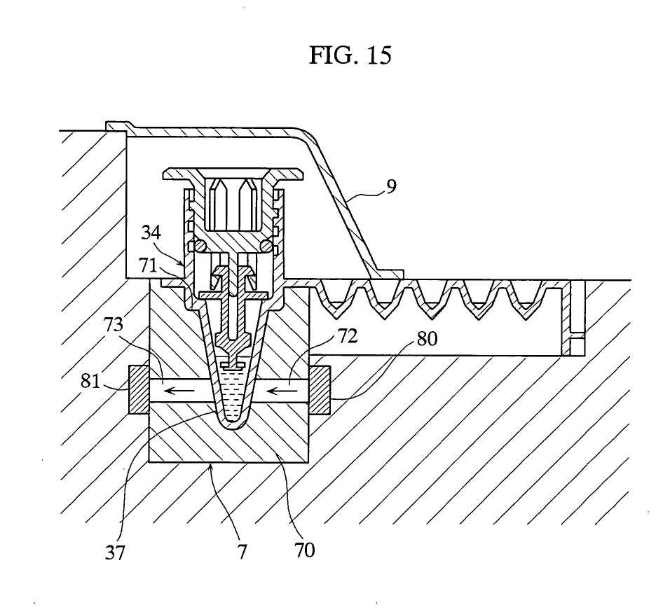

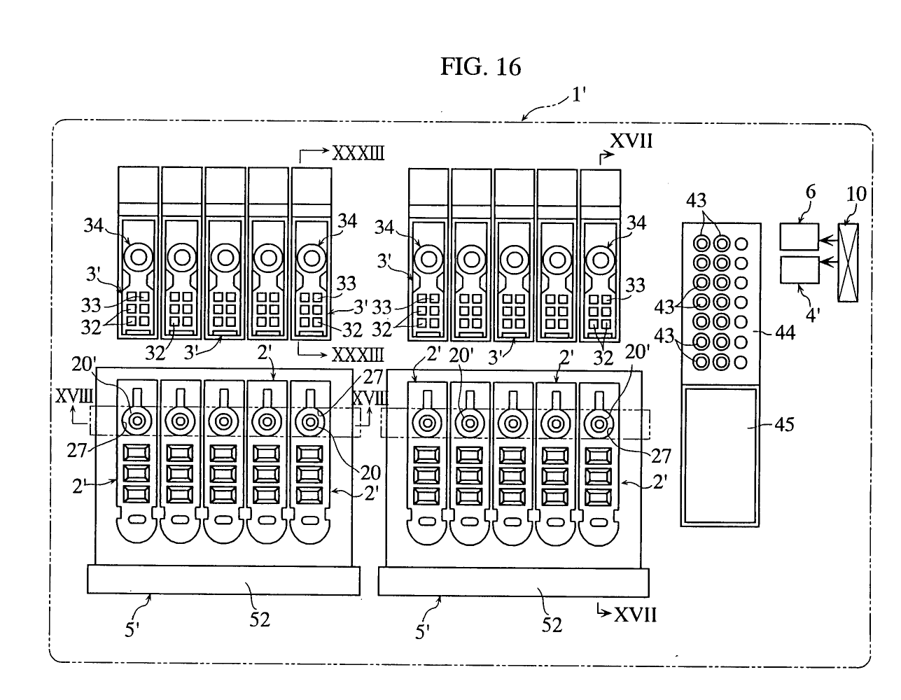

Now, the second embodiment of the present invention will be described with reference to FIGS. 16 through 33. In the drawings referred to below, similar constituents to those of the first embodiment of the present invention already described will be given the same numerals, and duplicating description thereof will not be repeated.

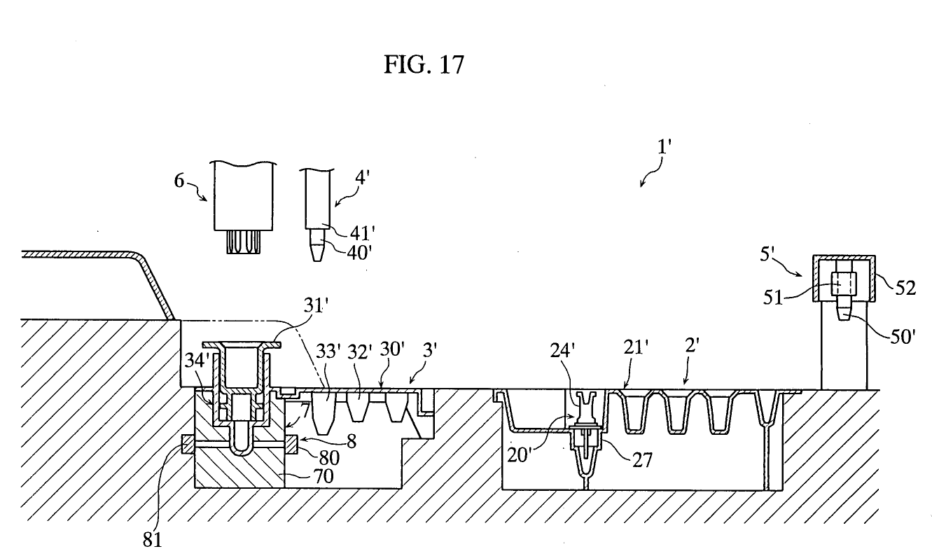

A nucleic acid analyzing apparatus 1′ shown in FIGS. 16 to 18 utilizes, like the foregoing nucleic acid analyzing apparatus 1 (refer to FIG. 1 and others), a plurality of nucleic acid purification cartridges 2′ and the same number of nucleic acid amplification cartridges 3′, and includes a pipet device 4′ and a nucleic acid purification mechanism 5′ as shown in FIG. 17.

As shown in FIG. 19, the nucleic acid purification cartridge 2′ serves to enable the automatic purification of the nucleic acid in the nucleic acid analyzing apparatus 1′, and includes a nucleic acid extracting element 20′ and a cartridge main body 21′.

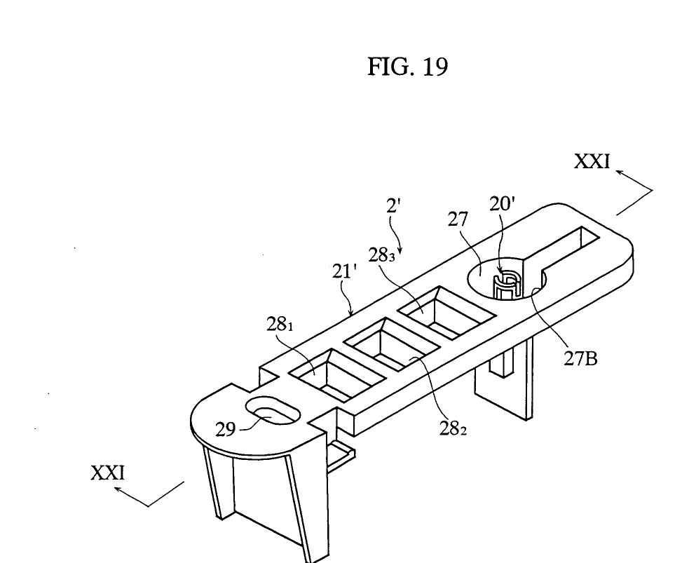

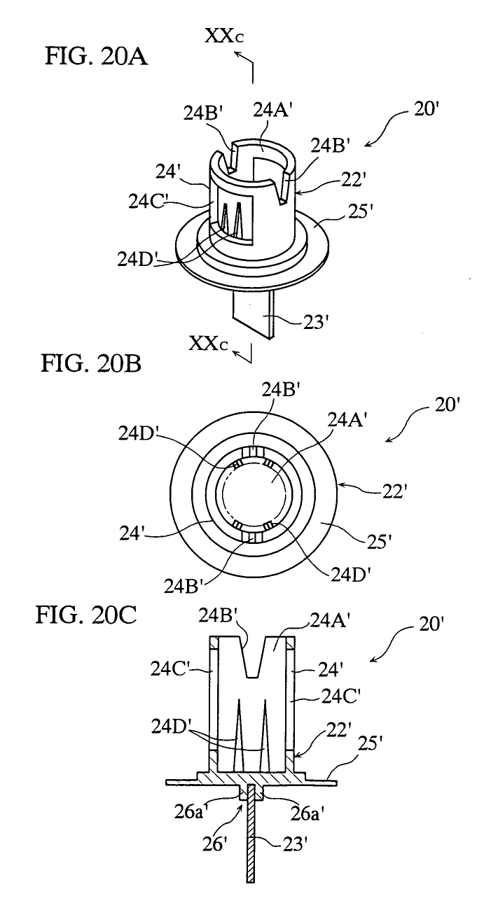

The nucleic acid extracting element 20′ serves to carry the nucleic acid contained in the specimen, and includes a retaining member 22′ and a solid matrix 23′ as explicitly shown in FIGS. 20A to 20C. The retaining member 22′ includes a cylindrical portion 24′, a flange 25′, and a holding portion 26′, and an entirety thereof is formed by, for example, a resin molding process.

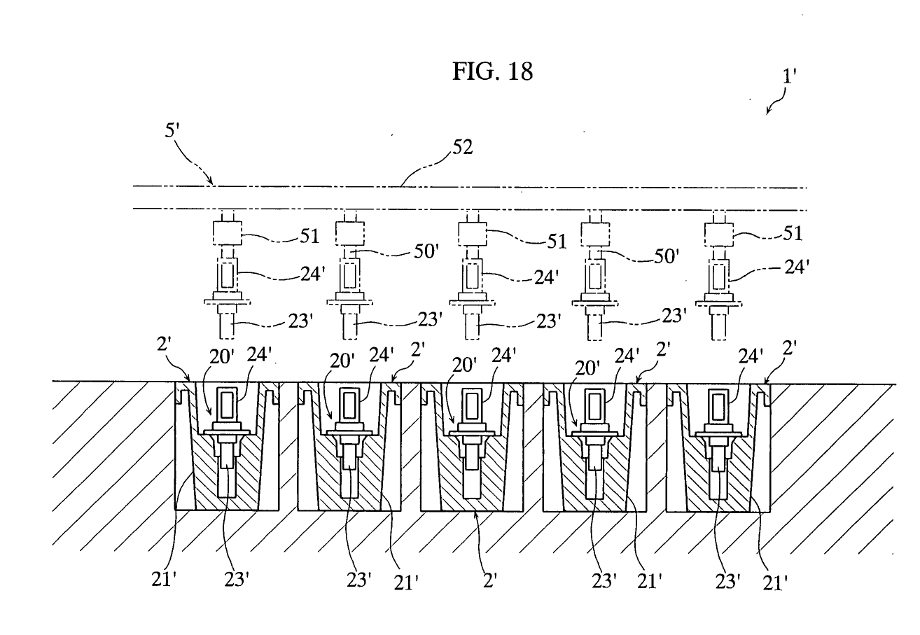

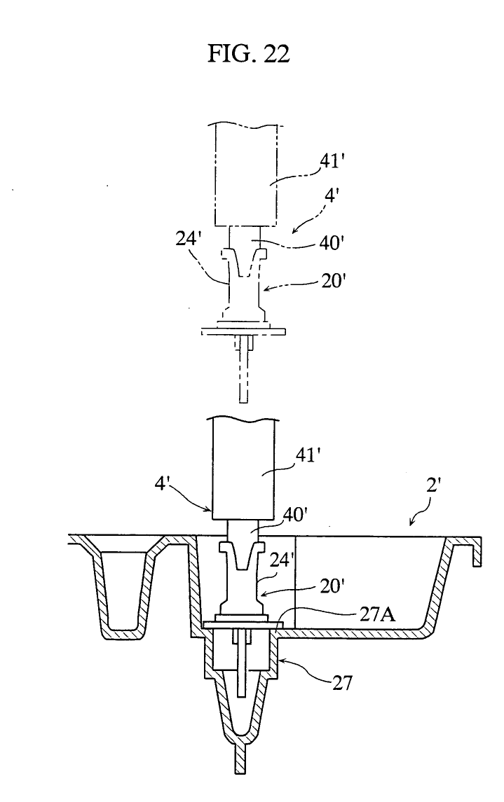

The cylindrical portion 24′ is utilized when moving the nucleic acid extracting element 20′ (refer to FIGS. 18 and 22), and includes a recessed portion 24A′, cutaway portions 24B′, 24C′ and a plurality of ribs 24D′. The recessed portion 24A′ is to be fitted to a tip portion 42′ of a nozzle 40′ of the pipet device 4′ (refer to FIGS. 26A and 26B) to be subsequently described, or with an insertion pin 50′ of a nucleic acid purification mechanism 5′, and is of a column shape. The cutaway portions 24B′, 24C′ serve to grant elasticity to the cylindrical portion 24′, and include a pair of V-shaped notches 24B′ and a rectangular through hole 24C′. Thus, the cutaway portions 24B′, 24C′ serve to apply, when the tip portion 42′ of the nozzle 40′ or the insertion pin 50′ is fitted to the recessed portion 24A′ (refer to FIGS. 18 and 22), an elastic force to those components to thereby enhance the engagement. The plurality of ribs 24D′ applies a frictional force to the tip portion 42′ of the nozzle 40′ or the insertion pin 50′ and the recessed portion 24A′ when they are fitted, to thereby enhance the engagement, and is disposed to vertically extend on an inner surface of the cylindrical portion 24′.

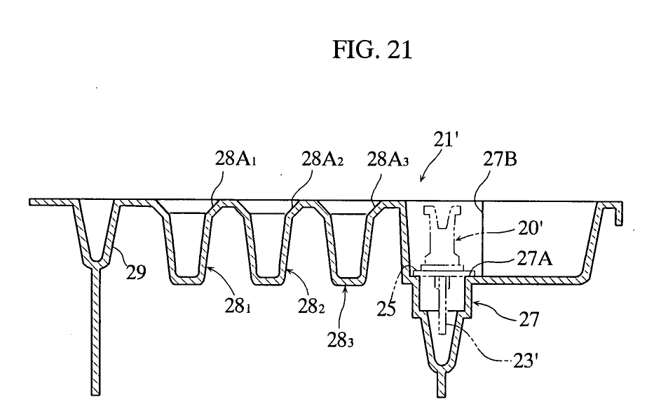

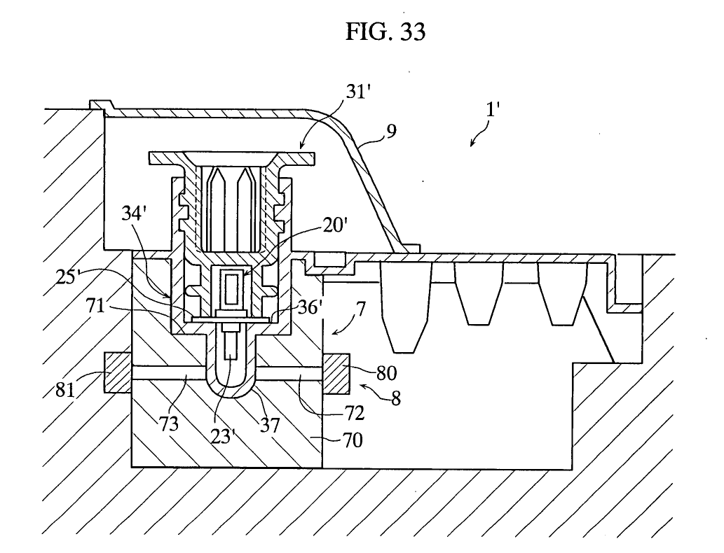

The flange 25′ is of a ring shape radially projecting outward. The flange 25′ is to be engaged, when the nucleic acid extracting element 20′ is retained at a target position (accommodation chamber 27 in the nucleic acid purification cartridge 2′ and a reactor 34′ in the nucleic acid amplification cartridge 3′), with stepped portions 27A, 36′ formed on the target position (refer to FIGS. 21 and 33).

The holding portion 26′ serves to hold an end portion of the solid matrix 23′ to unify the solid matrix 23′ with the retaining member 22′, and includes a pair of clips 26a′. It is preferable to form the pair of clips 26a′ to contact the solid matrix 23′ via a contact area as small as possible, in order to upgrade the collection efficiency of the nucleic acid. This is because it is difficult to elute the nucleic acid present in the contact area between the pair of clips 26a′ and the solid matrix 23′, in the process of eluting and collecting the nucleic acid which is executed after the nucleic acid is once stuck to the solid matrix 23′.

The solid matrix 23′ serves to carry the nucleic acid contained in the specimen, and is for example formed of a filter paper with a reagent for extracting the nucleic acid provided thereon. The solid matrix 23′ is of a strip shape, and an end portion thereof is to be held by the holding portion 26′, thus to be suspended by the retaining member 22′.

As shown in FIGS. 19 and 21A, the cartridge main body 21′ includes, as the foregoing cartridge main body 21 of the nucleic acid purification cartridge 2 (refer to FIGS. 5 and 6), the accommodation chamber 27, three cleaner wells 28₁ to 28₃, and a specimen well 29, while the surplus liquid removal chamber 21A (refer to FIGS. 5 and 6) is omitted. Naturally, the cartridge main body 21′ may also include the surplus liquid removal chamber.

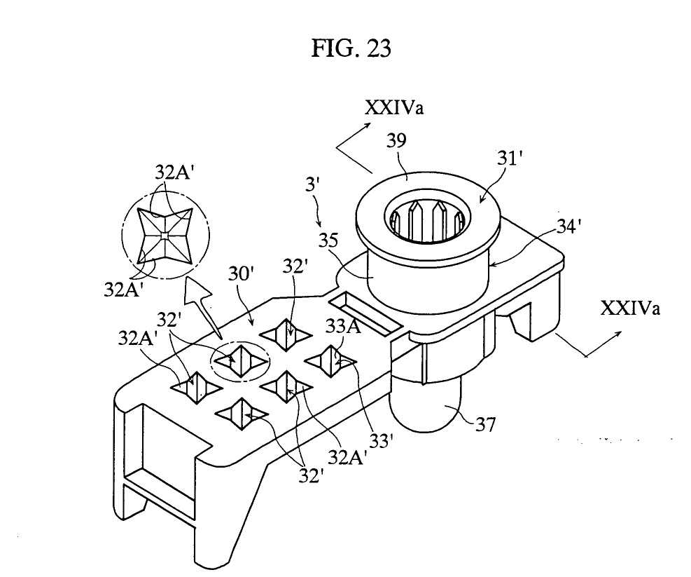

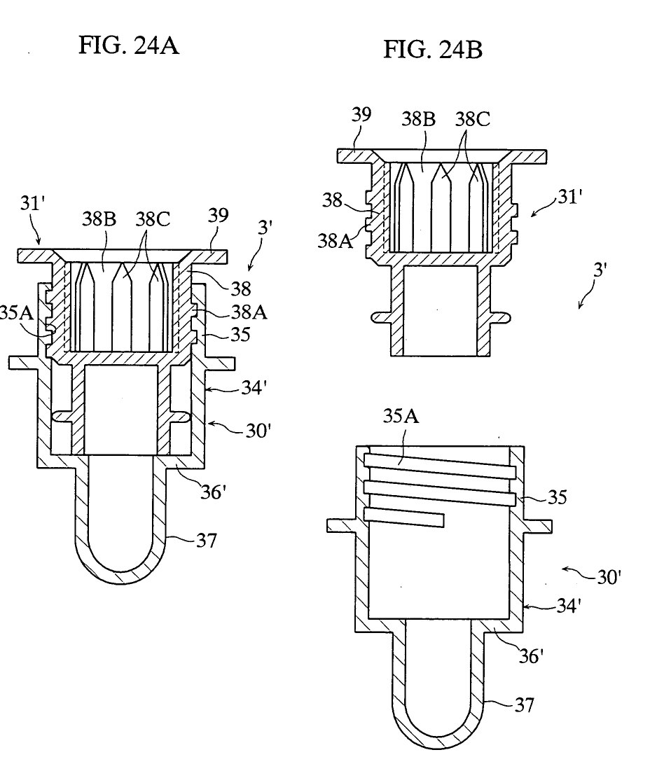

As shown in FIGS. 23, 24A and 24B, the nucleic acid amplification cartridge 3′ serves to enable the nucleic acid analyzing apparatus 1 to execute the automatic amplification and measurement of the nucleic acid, and includes a cartridge main body 30′ and the cap 31′.

The cartridge main body 30′ includes five reagent wells 32′, a mixing well 33′, and a reactor 34′, and the wells 32′, 33′, 34′ are integrally formed for example by a resin molding process.

The reagent wells 32′ serve to retain the reagent necessary for the amplification and measurement of the nucleic acid, in a form of a solution or suspension. Each of the reagent wells 32′ has a generally rectangular horizontal cross-section, however more precisely, the four sides 32A′ each have an inwardly protruding central portion. Accordingly, the four corners of the reagent wells 32′ define an acute angle narrower than 90 degrees. Such configuration prevents the reagent from remaining stuck to a lateral surface 32A′ of the reagent well 32′, thereby concentrating the reagent on a bottom portion of the reagent well 32′. This allows effectively utilizing the reagent retained in the reagent well 32′, and reducing the amount of the reagent to be loaded in the reagent well 32′ when the reagent is an expensive one, thus reducing the manufacturing cost. Such effect is also obtainable by forming grooves or ribs on the lateral surface 32A′ of the reagent well 32′.

Here, the types of the reagent to be loaded in the reagent wells 32′ are selected in accordance with the amplification method or measurement method to be adopted. Applicable amplification methods include the PCR process, the ICAN process, the LAMP process and the NASBA process.

The mixing well 33′ is utilized to mix two or more reagents loaded in the reagent wells 32′, before supplying the reagents to the reactor 34′. The mixing well 33′ also has four corners formed in an acute angle narrower than 90 degrees as the foregoing reagent well 32′. Naturally, the mixing well 33′ may be provided with grooves or ribs on the lateral surface 33A′.

The reactor 34′ serves to accommodate the mixed reagent and the nucleic acid extracting element 20′, as well as to provide a location where the nucleic acid carried by the nucleic acid extracting element 20′ and the mixed reagent prepared in the mixing well 33′ are reacted (refer to FIG. 33). The reactor 34′ includes the cylindrical portion 35 and the reaction detecting portion 37, between which a stepped portion 36′ is provided. The stepped portion 36′ is to be engaged with the flange 25′ of the nucleic acid extracting element 20′ (refer to FIG. 33), and formed by reducing the diameter of the reaction detecting portion 37 with respect to that of the cylindrical portion 35.

The cap 31′ is utilized to select whether the inside of the reaction detecting portion 37 is sealed, and is removably attachable to the reactor 34′ (cylindrical portion 35). More specifically, the cap 31′ is subjected to a rotational force, to select either being mounted on the cylindrical portion 35 or being completely separated from the cylindrical portion 35 (reactor 34′). The cap 31′ includes the cylindrical-shaped main body 38 and the flange 39, as the foregoing cap 31 of the nucleic acid amplification cartridge 3 (refer to FIG. 9). However, since the nucleic acid analyzing apparatus 1′ is configured to utilize the nozzle 40′ of the pipet device 4′ to move the nucleic acid extracting element 20′, the cap 31′ is not provided with the holder 36 (refer to FIGS. 7B and 9), which is provided on the cap 31 of the nucleic acid amplification cartridge 3.

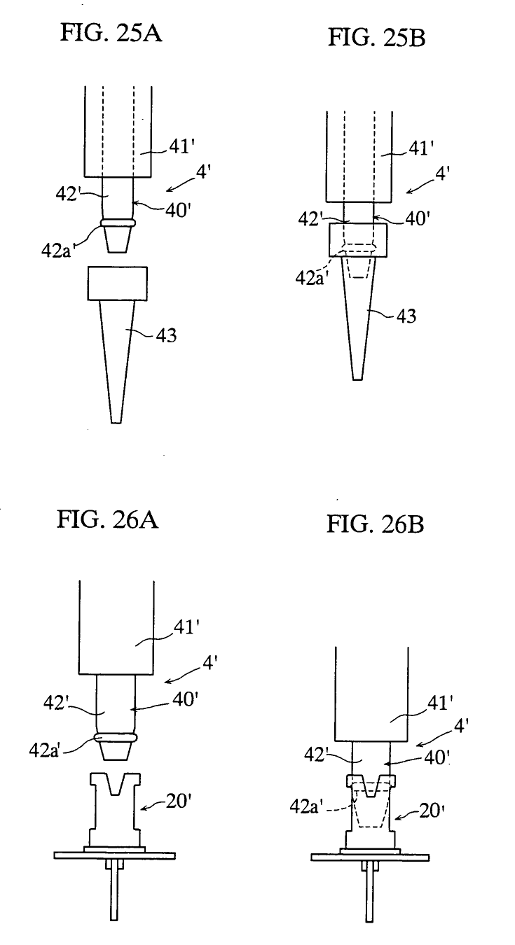

The pipet device 4′ shown in FIGS. 16 and 17 serves to prepare a mixed solution in the nucleic acid amplification cartridge 3, and to move the mixed solution to the reactor 34′. The pipet device 4′ includes the nozzle 40′ and a releasing member 41′, as shown in FIGS. 25 to 28.

The nozzle 40′ is configured to aspire and discharge a liquid and to move vertically and horizontally, to thereby move among the reagent well 32′, the mixing well 33′, and the reactor 34′ of the nucleic acid amplification cartridge 3′, and the accommodation chamber 27 of the nucleic acid purification cartridge 2′ (refer to FIGS. 16 and 17). When the mixed specimen is prepared or when the mixed specimen is dispensed to the reactor 34′ (reaction detecting portion 37), the chip 43 is attached to a tip portion 42′ of the nozzle 40′, as shown in FIGS. 25A and 25B. The nozzle 40′ is provided with an O-ring 42a′ fitted on the position on the tip portion 42′ where the chip 43 is to be attached, to achieve closer contact between the tip portion 42′ and the chip 43, when the chip 43 is attached to the tip portion 42′.

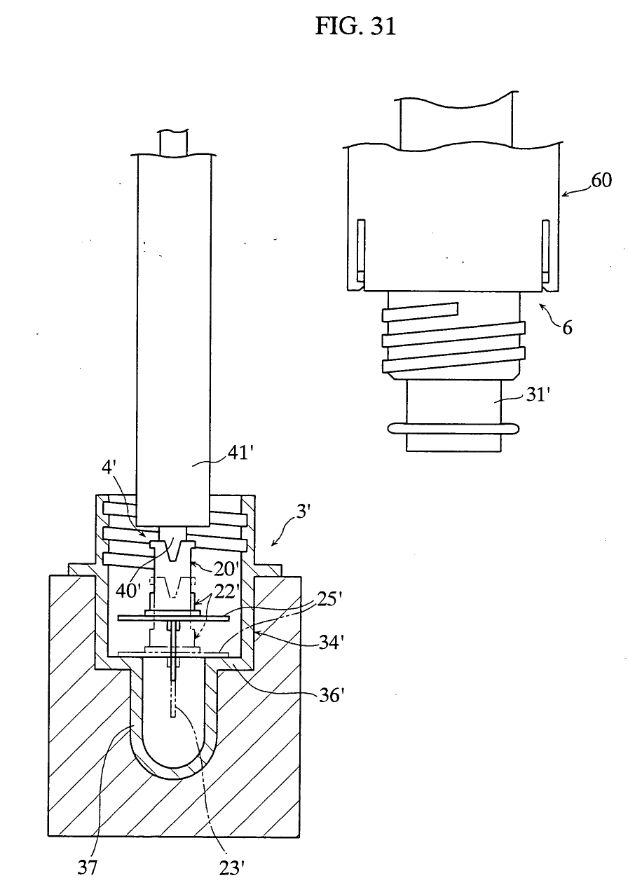

The pipet device 4′ further serves, as shown in FIG. 22, to take out the nucleic acid extracting element 20′ from the accommodation chamber 27 of the nucleic acid purification cartridge 2′, and to move the nucleic acid extracting element 20′ to the reactor 34′ of the nucleic acid amplification cartridge 3′, as shown in FIG. 31. When the pipet device 4′ thus works, the nucleic acid extracting element 20′ is attached to the tip portion 42′ of the nozzle 40′, as shown in FIGS. 26A and 26B.

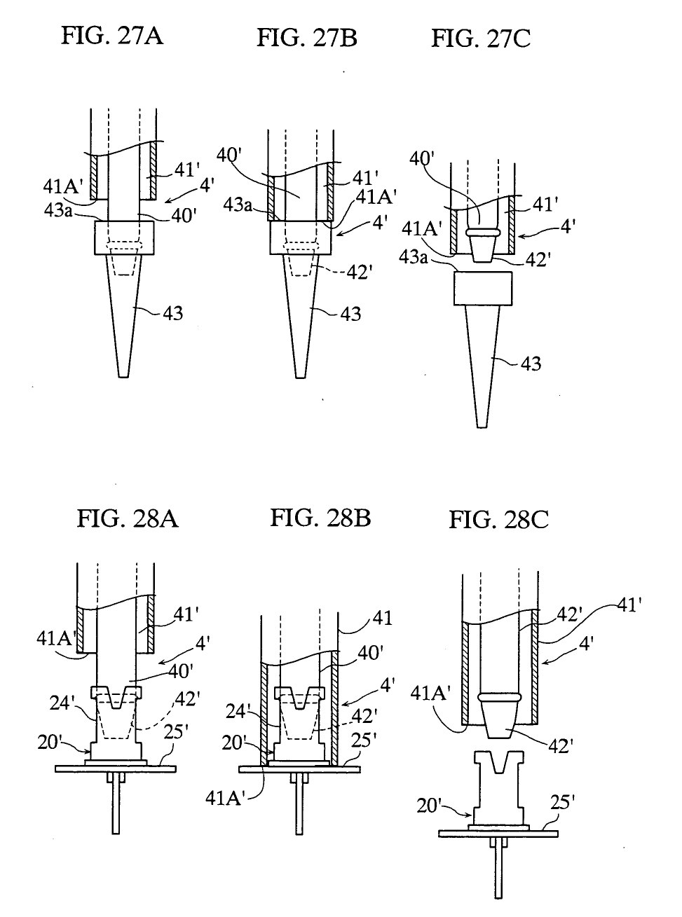

As shown in FIGS. 27 and 28, the releasing member 41′ serves to remove the chip 43 or the nucleic acid extracting element 20′ attached to the tip portion 42′ of the nozzle 40′. The releasing member 41′ encloses the nozzle 40′ to vertically move independently from the nozzle 40′. In other words, the releasing member 41′ is located above and end face 43a of the chip 43, or the flange 25′ of the nucleic acid extracting element 20′ (standby position) except when removing the chip 43 or the nucleic acid extracting element 20′ from the tip portion 42′ of the nozzle 40′, and is relatively moved downward with respect to the nozzle 40′, when removing the chip 43 or the nucleic acid extracting element 20′. When the releasing member 41′ is moved downward from the standby position by a predetermined distance, an end face 41A′ of the releasing member 41′ interferes with the end face 43a of the chip 43 or the flange 25′ of the nucleic acid extracting element 20′, thereby exerting a downward force to the chip 43 or the nucleic acid extracting element 20′. Thus, the chip 43 or the nucleic acid extracting element 20′ is removed from the tip portion 42′ of the nozzle 40′.

As shown in FIGS. 16 to 18, the nucleic acid purification mechanism 5′ serves to control the movement of the nucleic acid extracting element 20′, when extracting the nucleic acid in the specimen with the nucleic acid extracting element 20′. The nucleic acid purification mechanism 5′ includes a plurality of insertion pins 50′, the cylindrical element 51 and the support frame 52, as the foregoing nucleic acid purification mechanism 5 of the nucleic acid analyzing apparatus 1 (refer to FIGS. 2 to 4). Here, the insertion pins 50′ are of a similar shape to the tip portion 42′ of the nozzle 40′, to be properly engaged with the cylindrical portion 24′ of the nucleic acid extracting element 20′.

Now, an operation of the nucleic acid analyzing apparatus 1′ will be described.

The nucleic acid analyzing apparatus 1′ automatically executes the purification, amplification and measurement of the nucleic acid with the nucleic acid purification cartridge 2′ and the nucleic acid amplification cartridge 3′ installed therein, and once conditions according to the number and types of the cartridges 2′, 3′ (purification method, amplification method, measurement method) are set, as shown in FIGS. 16 to 18.

As shown in FIG. 18, the purification of the nucleic acid is executed through moving the nucleic acid extracting element 20′ in the nucleic acid purification cartridge 2′, by the nucleic acid purification mechanism 5′. More specifically, firstly the insertion pins 50′ of the nucleic acid purification mechanism 5′ are fitted to the corresponding cylindrical portion 24′ of the nucleic acid extracting element 20′, so that the plurality of nucleic acid extracting elements 20′ becomes integrally movable. Under such state, the nucleic acid purification mechanism 5′ causes the solid matrix 23′ of the plurality of nucleic acid extracting elements 20′ to be dipped in the specimen, so that the nucleic acid in the specimen is stuck to the solid matrix 23′.

Finally, each solid matrix 23′ is sequentially dipped in the cleaning liquid retained in the three cleaner wells 28₁ to 28₃ (refer to FIG. 19). More specifically, the solid matrix 23′ is cleaned through repeatedly moving the solid matrix 23 up and down in the cleaner wells 28₁ to 28₃ (refer to FIG. 19), by the nucleic acid purification mechanism 5′. In this process, the nucleic acid purification mechanism 5′ is controlled such that the solid matrix 23′ is repeatedly dipped completely in the cleaning liquid and lifted up to a position above the surface of the cleaning liquid. Such cleaning method efficiently removes a foreign substance from the solid matrix 23′, thereby effectively preventing the disturbance by the foreign substance against the amplification of the nucleic acid is effectively prevented in the subsequent amplification process of the nucleic acid, thus upgrading the accuracy in the analysis of the nucleic acid.

Once the cleaning process is completed, the solid matrix 23′ may be blow-dried while still retained by the nucleic acid purification mechanism 5′. After the cleaning process (or the blow-drying process as the case may be) of the solid matrix 23′ is completed, the nucleic acid extracting element 20′ is removed from the insertion pin 50′, and accommodated back in the accommodation chamber 27 in the nucleic acid purification cartridge 2′ (refer to FIGS. 19 and 21).

Thus, since the nucleic acid purification cartridge 2′ is configured to cause a solid substance (nucleic acid extracting element) to carry the target nucleic acid, the target nucleic acid can be easily moved inside the nucleic acid analyzing apparatus 1. From such viewpoint, the structure of the nucleic acid purification cartridge 2′ is beneficial for automatically executing the analysis of the nucleic acid.

The nucleic acid amplification is executed through preparing the mixed reagent in the nucleic acid amplification cartridge 3′, dispensing the mixed reagent to each reactor 34′ of the nucleic acid amplification cartridge 3′, and then transferring the solid matrix 23′ carrying the nucleic acid to the reactor 34′ with the retaining member 22′. It is to be noted that, as shown in FIG. 33, once the mixed reagent and the solid matrix 23′ are both accommodated in the reactor 34′, the temperature of the heat block 70 is controlled according to the adopted amplification method, thus to control the temperature of the reactor 34′.

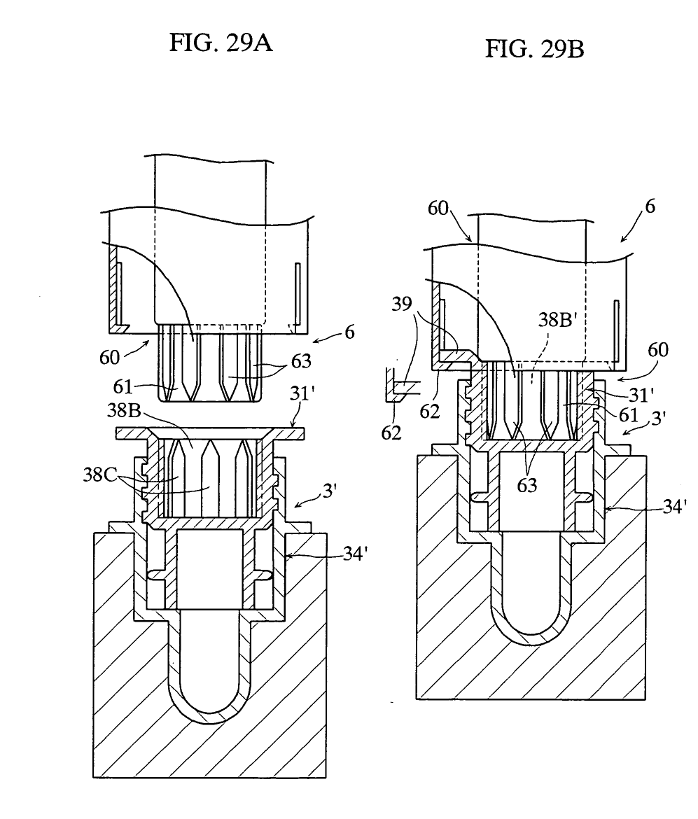

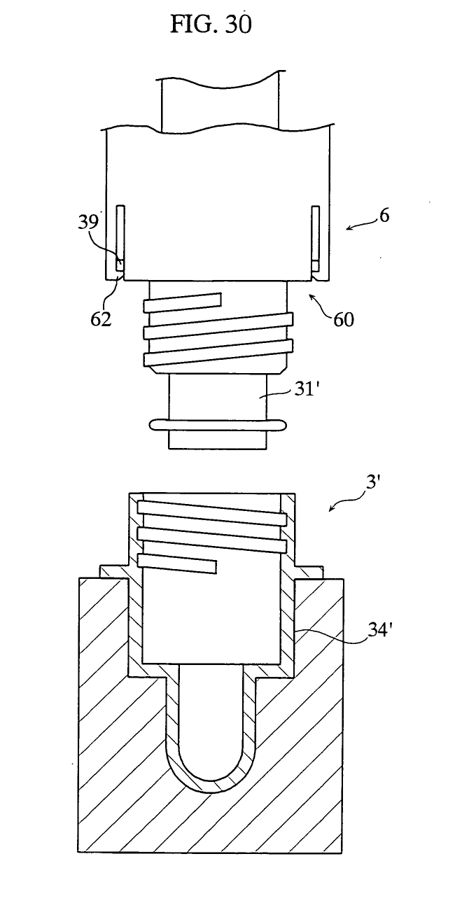

The preparation of the mixed reagent and the dispensing of the mixed solution to the reactor 34′ are executed, as in the foregoing nucleic acid analyzing apparatus 1 (refer to FIG. 1 and others), by controlling the movement of the pipet device 4′. Here, when the mixed reagent is dispensed to the reactor 34′ the cap 31′ has to be removed from the reactor 34′ by the cap attaching/removing mechanism 6 as shown in FIG. 31, which is executed, as shown in FIGS. 29 and 30, through inserting the rotating member 60 of the cap attaching/removing mechanism 6 into the recessed portion 38B′ of the cap 31′, and rotating the rotating member 60 thus to move the cap 31′ upward. When the rotating member 60 is inserted into the recessed portion 38B′, a pawl 62 of the rotating member 60 is engaged with the flange 39′ of the cap 31′, so that the cap 31′ removed from the reactor 34′ can be moved with the rotating member 60 and the rotating member 60. Thus in the nucleic acid analyzing apparatus 1′ and the nucleic acid amplification cartridge 3′, the configuration that allows easily and securely removing the cap 31′ from the nucleic acid amplification cartridge 3′ is achieved, to thereby attain the total automation of the nucleic acid amplification and the nucleic acid analysis.

Meanwhile, the transference of the solid matrix 23′ to the reactor 34′ is executed through a series of operations such as taking out the nucleic acid extracting element 20′ from the accommodation chamber 27 of the nucleic acid purification cartridge 2′ (refer to FIG. 22), transference of the nucleic acid extracting element 20′ to the reactor 34′ of the nucleic acid amplification cartridge 3′, and removal of the nucleic acid extracting element 20′ from the nozzle 40′ (refer to FIGS. 28 and 31).

The nucleic acid extracting element 20′ is taken out, as shown in FIG. 22, through locating the nozzle 40′ right above the accommodation chamber 27 of the nucleic acid purification cartridge 2′, moving the nozzle 40′ downward to engage the tip portion 42′ of the nozzle 40′ with the cylindrical portion 24′ of the nucleic acid extracting element 20′, and then moving the nozzle 40′ upward. Here, the cylindrical portion 24′ includes the cutaway portions 24B′, 24C′ namely the V-shaped notches 24B′ and the rectangular through hole 24C′ (refer to FIGS. 20A to 20C). Accordingly, when the tip portion 42′ of the nozzle 40′ is engaged with the cylindrical portion 24′, an appropriate elastic force can be exerted to the tip portion 42′. Consequently, the nucleic acid extracting element 20′ can be properly retained by the tip portion 42′ of the nozzle 40′, via the cylindrical portion 24′.

The nucleic acid extracting element 20′ can be moved by moving the nozzle 40′, with the nucleic acid extracting element 20′ retained by the tip portion 42′ of the nozzle 40′.

The nucleic acid extracting element 20′ can be removed, as shown in FIGS. 28 and 31, through locating the tip portion 42′ of the nozzle 40′ inside the reactor 34′ together with the nucleic acid extracting element 20′, and relatively moving the releasing member 41′ downward with respect to the nozzle 40′. When the releasing member 41′ is moved downward, the releasing member 41′ interferes with the flange 25′ of the nucleic acid extracting element 20′, thereby exerting a downward force to the flange 25′ and hence to the nucleic acid extracting element 20′, thus removing the nucleic acid extracting element 20′ from the tip portion 42′ of the nozzle 40′.

Thus, in the nucleic acid analyzing apparatus 1′, the nucleic acid extracting element 20′ can be moved utilizing the nozzle 40′ and the releasing member 41′, which are provided for preparing the specimen. Accordingly, the apparatus is prevented from becoming complicated despite aiming at executing the purification and amplification of the nucleic acid with a single apparatus, because of utilizing the mechanism that is indispensable any way (pipet device 4). Such configuration also suppresses an increase in number of mechanisms to be controlled, thus offering another advantage in suppressing an increase in dimensions of the apparatus.

As shown in FIG. 31, the nucleic acid extracting element 20′ removed from the tip portion 42′ of the nozzle 40′ is engaged with the stepped portion 36′ of the reactor 34′, via the flange 25′ of the retaining member 22′. At this stage, the solid matrix 23′ is accommodated in the reaction detecting portion 37 such that a lower end portion of the solid matrix 23′ is spaced by a predetermined distance from the bottom portion of the reaction detecting portion 37. Since the mixed reagent is already loaded in the reaction detecting portion 37, the entirety of the solid matrix 23′ is dipped, in the reaction detecting portion 37. Therefore, the nucleic acid is eluted from the solid matrix 23′, and the eluted nucleic acid is reacted with the reagent, thus to be amplified.

As stated above, the lower end portion of the solid matrix 23′ is spaced from the bottom portion of the reaction detecting portion 37. More precisely, the lower end portion of the solid matrix 23′ is at the level where the solid matrix 23′ is kept from interfering with the exciting light emitted by the photometric mechanism 8 to the reaction detecting portion 37 and the fluorescence to be measured (refer to FIG. 33). Such arrangement prevents, even when a solid carrier is employed for collecting the nucleic acid, the solid carrier from disturbing the measurement of the nucleic acid.

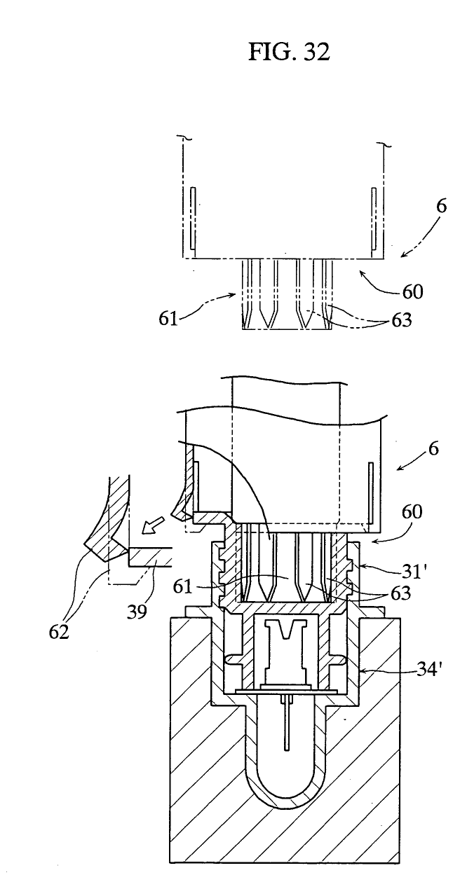

The measurement of the nucleic acid is executed by the photometric mechanism 8, with the cap 31′ of the reactor 34′ reattached thereto and with the reactor 34′ covered with the light-shielding member 9 disposed thereabove, as shown in FIGS. 32 and 33. The measurement of the nucleic acid by the photometric mechanism 8 is similarly executed to the process executed by the foregoing nucleic acid analyzing apparatus 1 (refer to FIG. 1 and others).

As described above, the nucleic acid analyzing apparatus 1 is, as the foregoing nucleic acid analyzing apparatus 1 (refer to FIG. 1 and others), capable of automatically analyzing the nucleic acid, simply by installing the set of the nucleic acid purification cartridge 2 and the nucleic acid amplification cartridge 3 configured as above. Accordingly, when executing the nucleic acid extraction and the nucleic acid amplification, installing the cartridges 2, 3 in the nucleic acid analyzing apparatus 1 is the only step that depends on the manual operation by the user. Such structure, therefore, significantly alleviates the burden on the user when executing the nucleic acid analysis, and minimizes the degradation in measurement reproducibility due to a difference in skill among the users which may create fluctuation in collection efficiency of the nucleic acid.

### WORKING EXAMPLES

Described below are the experiments carried out for examining, by SNP (Single Nucleotide Polymorphism) typing, whether the nucleic acid purification cartridge, the nucleic acid amplification cartridge and the nucleic acid analyzing apparatus according to the first embodiment of the present invention can properly purify and amplify human genome, adopted as the target nucleic acid.

### Working Example 1

### Formation of the Nucleic Acid Purification Cartridge

To form the nucleic acid purification cartridge, the cartridge main body (refer to 21 in the drawings) and the nucleic acid extracting element (refer to 20 in the drawings) were formed through the following method, after which the nucleic acid extracting element was accommodated in the accommodation chamber (refer to 27 in the drawings) of the cartridge main body, and a foam resin (foam urethane SAQ manufactured by Inoac Foam Company) employed as the water-absorbent material (refer to 21Ad, 21Ae in the drawings) was fixed to the surplus liquid removal chamber (refer to 21A in the drawings). The dimensions of the water-absorbent material 21Ad were 5 mm×8 mm×17 mm, and those of the water-absorbent material 21Ae were 5 mm×11 mm×14 mm.

The cartridge main body was formed in the shape as shown in FIGS. 5 and 6, by a resin molding process from PET.

The nucleic acid extracting element was formed by attaching the solid matrix (refer to 23 in the drawings) to the retaining member (refer to 22 in the drawings). The solid matrix was obtained by punching a FTA Classic Card (Cat. No. WB120205 manufactured by Whatman Japan, K.K.) to thereby form a disk of 2.5 mm in diameter. Here, the FTA Classic Card is a nucleic acid collecting paper mainly composed of cellulose. Meanwhile, the retaining member was formed in the shape as shown in FIGS. 7A and 7B, by a resin molding process from PET. However, immediately after the resin molding the retaining member was not yet provided with the stopper piece (refer to 26C in the drawings), and the stopper piece was formed, after opening a hole at the center of the solid matrix and inserting therethrough the pin-shaped portion (refer to 26B in the drawings) of the retaining member, by applying heat treatment to a tip portion of the pin-shaped portion. As already stated, the stopper piece serves to prevent the solid matrix from coming off from the pin-shaped portion.

### (Formation of the Nucleic Acid Amplification Cartridge)

To form the nucleic acid purification cartridge, the cartridge main body (refer to 30 in the drawings) and the cap (refer to 31 in the drawings) were formed in the shape as shown in FIGS. 8 and 9, by a resin molding process from PET, after which the cap was screw-fitted to the reactor (refer to 34 in the drawings) of the cartridge main body.

### (Purification of the Nucleic Acid)

For the nucleic acid purification, the specimen (refer to 29L in the drawings) was loaded in the specimen well (refer to 29 in the drawings) of the nucleic acid purification cartridge main body, and the cleaning liquid (refer to 28L₁ to 28L₃ in the drawings) was dispensed in the three cleaner wells (refer to 28₁ to 28₃ in the drawings), after which the nucleic acid purification cartridge was set in the nucleic acid analyzing apparatus (refer to 1 in the drawings) and the nucleic acid analyzing apparatus automatically executed the purification.

As the specimen, a whole blood (anticoagulant: containing heparin Na) was employed, in the dispensing amount of 120 μL. As the cleaning liquid 28L₁, cleaning liquid I (800 μL) shown in Table 1 given below was employed, cleaning liquid I (600 μL) shown in Table 1 as the cleaning liquid 28L₂, and cleaning liquid II (600 μL) shown in Table 1 as the cleaning liquid 28L₃.

TABLE 1

| | Composition | | pH |
|---|---|---|---|
| Cleaning Liquid I | 10 mM Tris-HCL | 1 mM EDTA | 8.0 |
| Cleaning Liquid II | 10 mM Tris-HCL | 0.1 mM EDTA | 8.0 |

On the part of the nucleic acid analyzing apparatus, the nucleic acid purification mechanism (refer to 5 in the drawings) was driven such that the nucleic acid extracting element (solid matrix) would move as described below.

Firstly, the insertion pin (refer to 50 in the drawings) of the nucleic acid operation mechanism was fitted to the cylindrical portion (refer to 24 in the drawings) of the retaining member, and the solid matrix was dipped in the whole blood in the specimen well. Then the solid matrix was cleaned in the three cleaner wells 28₁ to 28₃. When cleaning the solid matrix, the cleaner wells 28₁ to 28₃ were utilized by turns in the sequence of cleaner well 28₁→cleaner well 28₂→cleaner well 28₃. For cleaning the solid matrix in the cleaner well 28₁, the solid matrix 23 was moved up and down between a position where the solid matrix 23 is located above the surface of the cleaning liquid 28L₁ and a position where the solid matrix 23 is completely dipped in 28L₁, at a cycle of 20 Hz for one minute. For cleaning the solid matrix in the cleaner wells 28₂, 28₃, the same movement as with the cleaner well 28₁ was performed, except that the solid matrix 23 was moved up and down for two minutes.

Then surplus components that might disturb the nucleic acid amplification to be subsequently executed were eliminated. For eliminating the surplus components, the solid matrix and the tip portion of the retaining member (stopper piece, pin-shaped portion, tapered portion (refer to 26 in the drawings)) were pressed against the water-absorbent material (refer to 21Ad, 21Ae in the drawings).

### (Confirmation of Nucleic Acid Amplification)

The nucleic acid amplification was executed by the PCR process utilizing the mixed reagent solution A, B shown in Table 2 given below, and the extent of amplification of the nucleic acid was confirmed by the SNP (Single Nucleotide Polymorphism) typing of CYP2C19*2*3, which is a basic local alignment that codes a drug metabolic enzyme.

TABLE 2

| | |
|---|---|
| Mixed Reagent Solution A | 40 μL |

| | | |
|---|---|---|
| Sterilized Distilled Water | 35.6 | μL |
| 10 × Gene Taq Universal Buffer (Mg free) | 4 | μL |
| (Nippon Gene Co., Ltd.) | | |
| 5 units/μl Gene Taq FP | 0.4 | μL |

| | |
|---|---|
| Mixed Reagent Solution B | 40 μL |

| | | |
|---|---|---|
| Sterilized Distilled Water | 5.6 | μL |
| 10 × Gene Taq Universal Buffer (Mg free) | 4 | μL |
| (Nippon Gene Co., Ltd.) | | |
| 40% Glycerol Solution | 20 | μL |
| 100 mM MgCl₂ Solution (Nippon Gene Co., | 1.2 | μL |
| Ltd.) | | |
| 2.5 mM dNTP Mixture (Nippon Gene Co., | 6.4 | μL |
| Ltd.) | | |
| 100 μM CYP2C19*2 F-Primer (Sequence No. 1) | 0.4 | μL |
| 100 μM CYP2C19*2 R-Primer (Sequence No. 2) | 0.2 | μL |
| 100 μM CYP2C19*3 F-Primer (Sequence No. 3) | 0.2 | μL |
| 100 μM CYP2C19*3 R-Primer (Sequence No. 4) | 0.4 | μL |
| 5 μM CYP2C19*2 probe (Sequence No. 5) | 0.8 | μL |
| 5 μM CYP2C19*3 probe (Sequence No. 6) | 0.8 | μL |

Sequence No. 1: gttttctcttagatatgcaataattttccca
Sequence No. 2: cgagggttgttgatgtccatc
Sequence No. 3: gaaaaattgaatgaaaacatcaggattgta
Sequence No. 4: gtacttcagggcttggtcaata
Sequence No. 5: ttatgggttcccgggaaataatc-(BODIPY-FL)
Sequence No. 6: gcaccccctggatcc-(TAMRA)

More specifically, for confirming the amplification of the nucleic acid, the mixed reagent solution A or the mixed reagent solution B was individually dispensed to the reagent well (refer to 32₁, 32₂ in the drawings) of the nucleic acid amplification cartridge main body, after which the nucleic acid amplification cartridge was installed in the nucleic acid analyzing apparatus (refer to 1 in the drawings), so that the nucleic acid analyzing apparatus would automatically execute the confirmation.

In the nucleic acid analyzing apparatus, the pipet device (refer to 4 in the drawings), the cap attaching/removing mechanism (refer to 6 in the drawings), and the temperature control mechanism (refer to 7 in the drawings) were driven such that the nucleic acid extracting element (solid matrix) would move as described below.

After attaching the chip (refer to 43 in the drawings) to the nozzle (refer to 40 in the drawings) of the pipet device, 30 μL of mixed reagent solution A was collected from the reagent well 33A and 30 μL of mixed reagent solution B from the reagent well 33B, and both were dispensed to the mixing well (refer to 33 in the drawings). Then the nozzle was activated to aspire and discharge to agitate and mix the mixed reagent solution A, B thus to prepare the reaction solution, after which 50 μL of reaction solution was collected by the nozzle and dispensed to the reactor (refer to 34 in the drawings).

Meanwhile, after removing the cap (refer to 31 in the drawings) from the nucleic acid amplification cartridge by the rotating member (refer to 60 in the drawings) of the cap attaching/removing mechanism, the cap was moved to engage the engaging pawl (refer to 36A in the drawings) of the cap with the engaging head (refer to 24B in the drawings) of the nucleic acid extracting element, thus coupling them.

Then the cap and the nucleic acid extracting element 20 were accommodated in the reactor (refer to 34 in the drawings) of the nucleic acid amplification cartridge by the cap attaching/removing mechanism, and the rotating member was rotated thus to close the reactor with the cap. As a result, the solid matrix was sealed inside the reactor (refer to 34 in the drawings), being completely dipped in the reaction solution.

The heat block (refer to 70 in the drawings) of the temperature control mechanism was then activated to change the temperature of the reaction solution in the reactor, for the amplification of the target nucleic acid. The temperature was changed as 95° C. for 120 seconds, 50 cycles of 95° C. for 4 seconds and 54° C. for 60 seconds, 95° C. for 60 seconds, and 45° C. for 90 seconds.

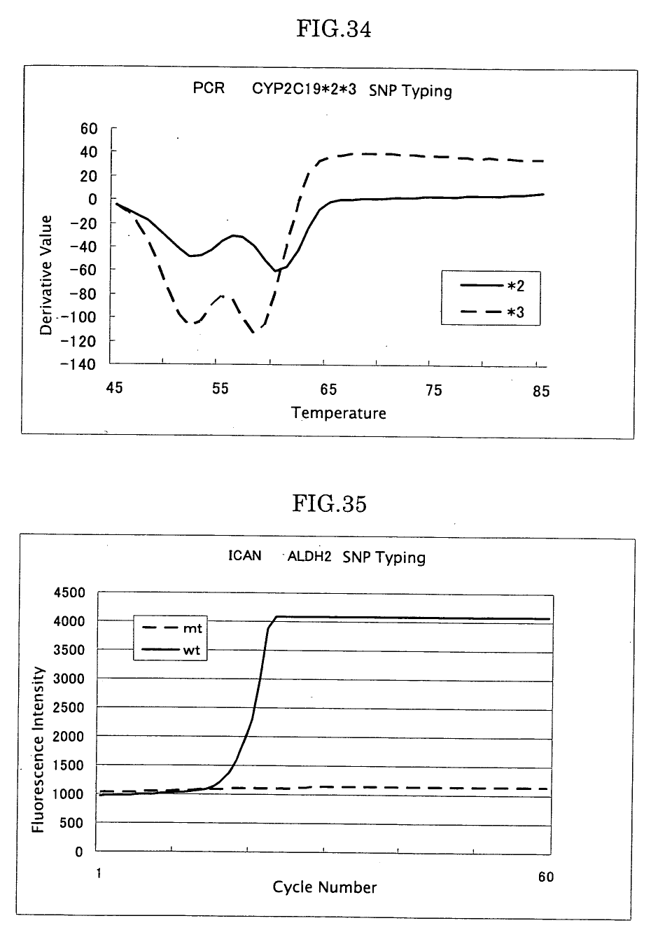

For the SNP typing, a Tm analysis was employed. To execute the Tm analysis, the temperature of reaction solution in which the nucleic acid was amplified was increased from 45° C. to 95° C. at a rate of 1° C./3 seconds, and transition of fluorescence intensity was measured at real time. Two measurement wavelengths of 515 to 555 nm (*2) and 585 to 750 nm (*3) were adopted, and the SNP typing was executed with respect to the respective measurement wavelengths (*2, *3). The measurement result of the fluorescence intensity at the respective wavelengths is shown in FIG. 34, in which the horizontal axis represents the temperature and the vertical axis a derivative value (change rate) of the fluorescence intensity.

As seen from FIG. 34, under the both measurement wavelengths *2, *3, the transition curves representing the derivative value (change rate) of the measured fluorescence intensity include two peaks. These peaks correspond to the wild-type SNP and mutant-type SNP, and therefore it is proven that the target nucleic acid was sufficiently amplified to enable identifying those types.

### Working Example 2

In this example, after purifying the nucleic acid in a similar process to Working Example 1, the ICNA process was employed for the amplification, and then the SNP typing was executed. As the amplification reagent, Cycleave ICAN human ALDH2 Typing Kit (Cat. No. CY101, manufactured by TaKaRa Bio Inc.) was employed, and the composition as shown in Table 3 was specified for the mixed reagent solution A, B to be loaded in the reagent well (refer to 32₁, 32₂ in the drawings) of the cartridge main body. The dispensing amount of the mixed reagent solution A, B, mixing conditions and the dispensing amount of the reaction solution were the same as those of Working Example 1.

TABLE 3

| | | |
|---|---|---|
| Mixed Reagent Solution A | 40 | μL |
| Sterilized Distilled Water | 15.2 | μL |
| 2× ICAN Reaction Buffer | 20 | μL |
| RNase H | 1.6 | μL |
| BcaBEST DNA Polymerase | 3.2 | μL |
| Mixed Reagent Solution B | 40 | μL |
| Sterilized Distilled Water | 13.6 | μL |
| 2× ICAN Reaction Buffer | 20 | μL |
| ALDH2 ICAN Primer Mix | 3.2 | μL |
| ALDH2 Probe Mix | 3.2 | μL |

### (Reaction Conditions)

For the reaction, the reaction solution with the solid matrix dipped therein was incubated at 70° C. for 300 seconds, and maintained at 60° C. for an hour. The reaction of one hour consisted of 60 cycles, each including 30 seconds of first step without measurement of the fluorescence intensity and 30 seconds of second step with measurement of the fluorescence intensity, and the fluorescence intensity was measured at real time. Two measurement wavelengths of 515 to 555 nm (mt) and 585 to 750 nm (wt) were adopted, and the SNP typing was executed with respect to the wild-type SNP and the mutant-type SNP. The measurement result of the fluorescence intensity at the respective wavelengths is shown in FIG. 35, in which the horizontal axis represents the number of cycles and the vertical axis the fluorescence intensity.

As seen from FIG. 35, after a certain number of cycles are performed, an increase in fluorescence intensity corresponding to the mutant-type SNP is observed, while the fluorescence intensity corresponding to the wild-type SNP barely increases despite the progress of the cycles. From the result shown in FIG. 35, therefore, it is proven that the target nucleic acid (wild-type SNP) was selectively and sufficiently amplified to enable identifying the wild-type SNP and the mutant-type SNP.

### Working Example 3

In this example, after purifying the nucleic acid in a similar process to Working Example 1, the LAMP process was employed for the amplification, and then the SNP typing was executed. As the amplification reagent, Loopamp P450 typing reagent kit (CYP2C9*3, manufactured by Eiken Chemical Co., Ltd.) was employed, and the composition as shown in Table 3 was specified for the mixed reagent solution A, B to be loaded in the reagent well (refer to 33A, 33B in the drawings) of the cartridge main body. The dispensing amount of the mixed reagent solution A, B, mixing conditions and the dispensing amount of the reaction solution were the same as those of Working Example 1.

TABLE 4

| | | |
|---|---|---|
| Mixed Reagent Solution A | 40 | μL |
| Sterilized Distilled Water | 9.6 | μL |
| Reaction Mix. SNP | 16 | μL |
| Fluorescent Detection Reagent for Genome | 3.2 | μL |
| 10 mM Tris Solution: PH 8.0 | 8 | μL |
| Bst DNA Polymerase | 3.2 | μL |
| Mixed Reagent Solution B | 40 | μL |
| Sterilized Distilled Water | 11.2 | μL |
| Reaction Mix. SNP | 16 | μL |
| Primer Mix. for 2C9*3 (C) | 12.8 | μL |
| or | | |
| Primer Mix. for 2C9*3 (A) | | |

### (Reaction Conditions)

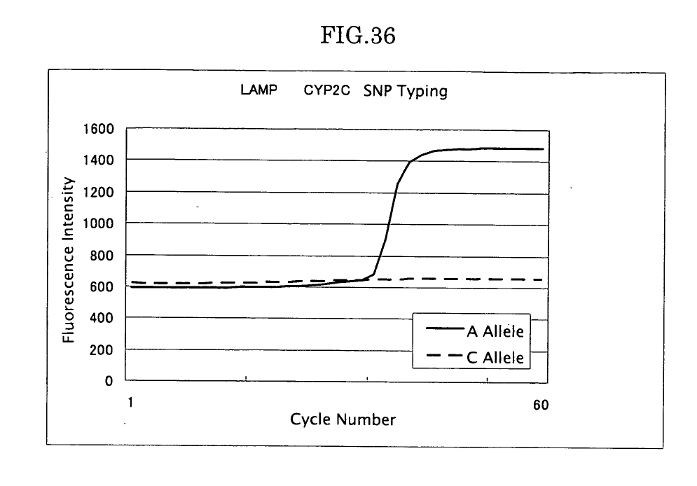

For the reaction, the reaction solution with the solid matrix dipped therein was processed at 95° C. for 5 minutes, and maintained at 60° C. for an hour. The reaction of one hour consisted of 60 cycles, each including 30 seconds of first step without measurement of the fluorescence intensity and 30 seconds of second step with measurement of the fluorescence intensity, and the fluorescence intensity was measured at real time during the second step each cycle, at the measurement wavelength of 515 to 555 nm. The measurement result of the fluorescence intensity during the second step each cycle is shown in FIG. 36, in which the horizontal axis represents the number of cycles and the vertical axis the fluorescence intensity.

As seen from FIG. 36, after a certain number of cycles are performed, an increase in fluorescence intensity corresponding to the mutant-type SNP (A allele in the graph) is observed, while the fluorescence intensity corresponding to the wild-type SNP (G allele in the graph) barely increases despite the progress of the cycles. From the result shown in FIG. 36, therefore, it is proven that the target nucleic acid (wild-type SNP) was selectively and sufficiently amplified to enable identifying the wild-type SNP and the mutant-type SNP.

As is understood from the results of Working Examples 1 to 3, employing the nucleic acid extracting element according to the first embodiment of the present invention for purification of the nucleic acid allows properly executing the amplification of the target nucleic acid, not only when the amplification is executed by the PCR process, but also by the ICAN process or LAMP process. In other words, it is proven that employing the nucleic acid purification cartridge, the nucleic acid extraction cartridge and the nucleic acid analyzing apparatus according to the first embodiment of the present invention enables automatically analyzing the nucleic acid. The present invention, therefore, significantly alleviates the burden imposed on the user in the series of operations including the nucleic acid purification, nucleic acid amplification and nucleic acid measurement, improves the analysis efficiency, and also suppresses an increase in dimensions of the apparatus and in manufacturing cost thereof.

Although the structure according to the first embodiment of the present invention was employed in Working Examples 1 to 3 for examining whether the nucleic acid was properly amplified, it is certain that the structure according to the second embodiment of the present invention can also properly amplify the nucleic acid, thereby equally providing the foregoing advantageous effects.

1. A nucleic acid amplification container to be set in a nucleic acid analyzing apparatus, the container comprising:
a container main body including a reactor in which a target nucleic acid is to be reacted with an amplification reagent; and
a cap that covers an upper opening of the reactor and is removably attached to the container main body.

2. The nucleic acid amplification container according to claim 1, wherein the cap is thread-engageable with the reactor, the cap being attached to and detached from the reactor by being rotated.

3. The nucleic acid amplification container according to claim 2, wherein the nucleic acid analyzing apparatus includes a rotating member that applies rotational force to the cap, and wherein the cap includes an engaging portion to be engaged with the rotating member to enable the rotating member to exert the rotational force.

4. The nucleic acid amplification container according to claim 3, wherein the engaging portion includes a column-shaped recessed portion in which the rotating member is inserted, and
wherein the recessed portion includes a plurality of vertically extending ribs circumferentially aligned on an inner circumferential surface at regular intervals.

5. The nucleic acid amplification container according to claim 4, wherein the rib has a reducing width toward an upper end portion thereof.

6. The nucleic acid amplification container according to claim 3, wherein the cap includes a projection via which the rotating member retains the cap.

7. The nucleic acid amplification container according to claim 6, wherein the projection is an outwardly projecting flange.

8. A nucleic acid preparation kit to be set in a nucleic acid analyzing apparatus, the kit comprising:
a nucleic acid extracting container for extracting a target nucleic acid from a specimen; and a nucleic acid amplification container that amplifies the target nucleic acid;
wherein the nucleic acid amplification container comprises: a container main body including a reactor in which the target nucleic acid is to be reacted with an amplification reagent; and a cap that covers an upper opening of the reactor and is removably attached to the container main body.

9. The nucleic acid preparation kit according to claim 8, wherein the cap is thread-engageable with the reactor, the cap being attached to and detached from the reactor by being rotated.

10. The nucleic acid preparation kit according to claim 9, wherein the nucleic acid analyzing apparatus includes a rotating member that applies rotational force to the cap, and wherein the cap includes an engaging portion to be engaged with the rotating member to enable the rotating member to exert the rotational force.

11. The nucleic acid preparation kit according to claim 10, wherein the engaging portion includes a column-shaped recessed portion in which the rotating member is inserted, and the recessed portion includes a plurality of vertically extending ribs circumferentially aligned on an inner circumferential surface at regular intervals.

12. The nucleic acid preparation kit according to claim 11, wherein the rib has a reducing width toward an upper end portion thereof.

13. The nucleic acid preparation kit according to claim 10, wherein the cap includes a projection via which the rotating member retains the cap.

14. The nucleic acid preparation kit according to claim 13, wherein the projection is an outwardly projecting flange.

15. The nucleic acid preparation kit according to claim 8, wherein the nucleic acid extracting container includes a nucleic acid extracting element that extracts the target nucleic acid from the specimen and carries the extracted nucleic acid, and a container main body formed as a separate body from the nucleic acid extracting element and including an accommodation chamber that stores therein the nucleic acid extracting element.

16. The nucleic acid preparation kit according to claim 15, wherein the nucleic acid extracting element and the cap are provided with a retaining device that causes the cap to retain the nucleic acid extracting element cap to integrally move the nucleic acid extracting element with the cap.

17. The nucleic acid preparation kit according to claim 16, wherein the retaining device includes a protruding or recessed portion for engagement provided on one of the nucleic acid extracting element and the cap, and one or more engaging pawls provided on the other of the nucleic acid extracting element and the cap, to be engaged with the protruding or recessed portion for engagement.

18. The nucleic acid preparation kit according to claim 16, wherein the nucleic acid extracting element and the cap are provided with a guide mechanism that delimits a position of the cap with respect to the nucleic acid extracting element, when the cap is caused to retain the nucleic acid extracting element.

19. The nucleic acid preparation kit according to claim 18, wherein the guide mechanism includes a pin provided on one of the nucleic acid extracting element and the cap, and an insertion hole provided on the other of the nucleic acid extracting element and the cap, for the pin to be inserted therein.

20. The nucleic acid preparation kit according to claim 15, wherein the nucleic acid extracting element includes a solid matrix that carries the target nucleic acid, and a retaining member that retains the solid matrix.

21. The nucleic acid preparation kit according to claim 20, wherein the solid matrix is retained in an inclined orientation with respect to a vertical axis of the retaining member.

22. The nucleic acid preparation kit according to claim 21, wherein the solid matrix is retained in a horizontal or generally horizontal orientation with respect to the vertical axis.

23. The nucleic acid preparation kit according to claim 21, wherein the solid matrix is pierced with the retaining member to be retained by the retaining member.

24. The nucleic acid preparation kit according to claim 23, wherein the retaining member includes a tapered portion with a reducing diameter toward an end portion, a pin-shaped portion extending from the tapered portion to penetrate through the solid matrix, and a stopper piece that restricts the solid matrix from coming off from the pin-shaped portion.

25. The nucleic acid preparation kit according to claim 21, wherein the solid matrix is of a disk shape.

26. The nucleic acid preparation kit according to claim 20, wherein the solid matrix is of a sheet shape, and retained by the retaining member being suspended therefrom.

27. The nucleic acid preparation kit according to claim 26, wherein the retaining member includes a holder that holds an end portion of the solid matrix to suspend the solid matrix.

28. The nucleic acid preparation kit according to claim 20, wherein the retaining member includes a projection, and
wherein the reactor includes a stepped portion to be engaged with the projection.

29. The nucleic acid preparation kit according to claim 28, wherein in the case where the nucleic acid analyzing apparatus includes a transferring member that takes out the nucleic acid extracting element from the accommodation chamber and transfers the nucleic acid extracting element to the reactor,
wherein the retaining member includes an engaging portion to be engaged with the transferring member, and
wherein the projection can be utilized for releasing the engagement of the transferring member and the retaining member.

30. The nucleic acid preparation kit according to claim 29, wherein in the case where the nucleic acid analyzing apparatus includes a cylindrical member that encloses the transferring member and is relatively movable in a vertical direction with respect to the transferring member,
wherein the projection is subjected to a downward force when the cylindrical member is relatively moved downward with respect to the transferring member and thereby interferes with the projection.

31. The nucleic acid preparation kit according to claim 30, wherein the projection is an outwardly projecting flange.

32. The nucleic acid preparation kit according to claim 20, wherein the nucleic acid amplification container is disposed such that the solid matrix is spaced from a bottom portion of the reactor when the nucleic acid extracting element is taken out of the accommodation chamber and accommodated in the reactor.

33. The nucleic acid preparation kit according to claim 20, wherein the retaining member includes a sealing member that defines a sealed space in the reactor, when the nucleic acid extracting element is accommodated in the reactor while being retained by the cap, and
wherein the sealing member is fixed at an upper position than where the solid matrix is retained.

34. The nucleic acid preparation kit according to claim 8, wherein the nucleic acid extracting container further includes one or more cleaner wells that store therein a cleaning liquid for removing impurity other than the target nucleic acid from the nucleic acid extracting element, and
wherein the nucleic acid amplification container further includes one or more reagent wells that store therein a reagent necessary for amplifying the target nucleic acid.

35. A nucleic acid amplification apparatus arranged to cooperate with a nucleic acid amplification container, wherein the container comprises: a container main body including a reactor in which the target nucleic acid is to be reacted with an amplification reagent; and a cap that covers an upper opening of the reactor and is removably attached to the container main body.

36. The nucleic acid analyzing apparatus according to claim 35, further comprising a cap attaching/removing device that attaches and removes the cap.

37. The nucleic acid analyzing apparatus according to claim 36, wherein the nucleic acid amplification container is configured to employ the cap that is screw-engaged with the reactor, so that exerting a rotational force to the cap allows attaching and removing the cap to and from the reactor, and
wherein the cap attaching/removing device includes a rotating member that exerts the rotational force to the cap.

38. The nucleic acid analyzing apparatus according to claim 37, wherein the nucleic acid amplification container is configured to employ the cap that includes an engaging portion having a column-shaped recessed portion in which a tip portion of the rotating member is inserted, and a plurality of vertically extending ribs circumferentially aligned at regular intervals on an inner circumferential surface of the recessed portion, and
wherein the rotating member includes a plurality of protrusions to be located between adjacent ones of the plurality of ribs of the cap when the tip portion is inserted in the recessed portion.

39. The nucleic acid analyzing apparatus according to claim 38, wherein the plurality of protrusions is disposed to vertically extend, with a reducing width toward a lower end portion.

40. The nucleic acid analyzing apparatus according to claim 37, wherein the nucleic acid amplification container is configured to employ the cap that includes a projection formed to project outward, and
wherein the cap attaching/removing device includes an engaging pawl to be engaged with the projection, and can move the cap at least in a vertical direction, with the engaging pawl being engaged with the projection.

41. A nucleic acid analyzing apparatus for use with a nucleic acid extracting container and a nucleic acid amplification container to prepare a target nucleic acid from a specimen and to analyze the target nucleic acid,
wherein the nucleic acid amplification container comprises: a container main body including a reactor that provides a space for amplifying the target nucleic acid with a nucleic acid extracting element retaining the target nucleic acid extracted from the specimen; and a cap that covers an upper opening of the reactor.

42. The nucleic acid analyzing apparatus according to claim 41, further comprising a cap attaching/removing device that attaches and removes the cap.

43. The nucleic acid analyzing apparatus according to claim 42, wherein the nucleic acid amplification container is configured to employ the cap that is screw-engaged with the reactor, so that exerting a rotational force to the cap allows attaching and removing the cap to and from the reactor, and
wherein the cap attaching/removing device includes a rotating member that exerts the rotational force to the cap.

44. The nucleic acid analyzing apparatus according to claim 42, wherein in the case where the cap is set to retain the nucleic acid extracting element,
wherein the cap attaching/removing device operates to move the cap taken out of the reactor, cause the cap to retain the nucleic acid extracting element retained in the accommodation chamber, thereby taking out the nucleic acid extracting element from the accommodation chamber and moving the cap with the nucleic acid extracting element to accommodate the nucleic acid extracting element in the reactor, and then to cover the upper opening of the reactor with the cap.

45. The nucleic acid analyzing apparatus according to claim 44, wherein in the case where the nucleic acid amplification container is configured to employ the cap that includes the recessed portion and the flange,
wherein the cap attaching/removing device includes a fitting element to be fitted in the recessed portion, and a cylindrical element that encloses the fitting element and includes a pawl portion to be engaged with the flange.

46. The nucleic acid analyzing apparatus according to claim 43, comprising a transferring member that takes out the nucleic acid extracting element from the accommodation chamber and transfers the nucleic acid extracting element to the reactor.

47. The nucleic acid analyzing apparatus according to claim 46, further comprising a cylindrical member that encloses the transferring member and is relatively movable in a vertical direction with respect to the transferring member,
wherein the cylindrical member removes the nucleic acid extracting element coupled with the transferring member, when moved downward with respect thereto.

48. The nucleic acid analyzing apparatus according to claim 47, further comprising a control unit that controls a movement of the transferring member and the cap attaching/removing device,
wherein the control unit executes:
a step of causing the rotating member retaining the cap to retreat from right above the reactor after removing the cap from the reactor with the rotating member;
a step of causing the transferring member to take out the nucleic acid extracting element from the accommodation chamber and to transfer the nucleic acid extracting element into the reactor;
a step of causing the cylindrical member to remove the nucleic acid extracting element from the transferring member and accommodating the nucleic acid extracting element in the reactor; and
a step of causing the rotating member to attach the cap to the reactor.

49. The nucleic acid analyzing apparatus according to claim 46, wherein in the case of employing the nucleic acid amplification container including a plurality of reagent wells that store therein a plurality of reagents necessary for amplification of the target nucleic acid,
wherein the transferring member is a nozzle used for dispensing or mixing the plurality of reagents in the nucleic acid amplification container.

50. The nucleic acid analyzing apparatus according to claim 49, wherein the nozzle is configured to aspire and discharge a liquid with a chip mounted thereon, and to take out the nucleic acid extracting element from the accommodation chamber when the chip is not mounted.

51. The nucleic acid analyzing apparatus according to claim 50, wherein the chip is mounted on the nozzle when a tip portion thereof is fitted to the chip, and fitting the tip portion to a recessed portion provided on the nucleic acid extracting element enables the nozzle to take out the nucleic acid extracting element from the accommodation chamber.

52. The nucleic acid analyzing apparatus according to claim 51, further comprising a cylindrical member that encloses the nozzle, and is relatively movable in a vertical direction with respect to the nozzle,
wherein the cylindrical member removes the chip or the nucleic acid extracting element fitted to the tip portion of the nozzle when moved downward with respect thereto.

53. The nucleic acid analyzing apparatus according to claim 52, wherein the nucleic acid extracting element includes a projection that interferes with the cylindrical member when the nucleic acid extracting element is removed from the nozzle.

54. The nucleic acid analyzing apparatus according to claim 50, wherein the nozzle is provided with an O-ring attached to the tip portion thereof, at a position to be fitted to the chip or the nucleic acid extracting element.
